(19) United States
(12) Patent Application Publication
(10) Pub. No.: US 2023/0232836 A1
(43) Pub. Date: Jul. 27, 2023

(54) MANNOSYLERYTHRITOL LIPID BIOLOGICAL PESTICIDES AND APPLICATIONS THEREOF

(71) Applicants: Mikros Biochem, Costa Mesa, CA (US); Advanced BioCatalytics Corporation, Irvine, CA (US)

(72) Inventors: Wenjie Xia, Irvine, CA (US); Larry Tung On Co, Lake Forest, CA (US)

(73) Assignees: Mikros Biochem, Costa Mesa, CA (US); Advanced BioCatalytics Corporation, Irvine, CA (US)

(21) Appl. No.: 18/159,664

(22) Filed: Jan. 25, 2023

(30) Foreign Application Priority Data
Jan. 25, 2022 (CN) 202210087291.7

(51) Int. Cl. A01N 63/32; A01P 3/00; C12P 19/44

(52) U.S. Cl. CPC A01N 63/32; A01P 3/00; C12P 19/44; C12P 2203/00; C12R 2001/645

(57) ABSTRACT

This disclosure describes biological pesticides that include biological mannosylerythritol lipids (MELs), and their application. Provided MEL-based pesticides are microbially produced by through microbial (fungal) fermentation of plant-based derivatives or plant-derived materials as a substrate. The biologically active components are obtained from multiple stage bio-processes including transformation, biochemical reaction, extraction, and other processing of raw materials. The synthesis, separation, concentration, purification, preparation of biological pesticides, and their application as a crop pathology treatment, are described. These biological pesticides may be used disease prevention and control for crops and other plants.

### CROSS-REFERENCE TO RELATED APPLICATION

This application claims priority to and the benefit of the earlier filing of China Application No. 202210087291.7, filed on Jan. 25, 2022, which is incorporated by reference herein in its entirety.

### FIELD OF THE DISCLOSURE

The present invention relates to a biopesticide prepared by a microbial method using plants, their derivatives or plant-derived materials as substrates, and is used in the field of plant disease prevention and control such as crops.

### BACKGROUND OF THE DISCLOSURE

Biological control refers to the use of one or one type of organism to inhibit another or another type of organism based on the competitive relationship between species, so as to achieve the purpose of controlling harmful organisms and protecting target organisms. Biological pesticides refer to pesticide formulations that use the organism itself or its secretions as raw materials and undergo synthetic processing to protect target crops from harmful organisms.

Compared with chemical agents, biological pesticides have many advantages, such as: (1) low toxicity and high efficiency, easy to be degraded by microorganisms in the environment, and friendly to the environment; (2) wide source of raw materials for production, and many ways of research and development and utilization; (3) it is not easy for pathogenic organisms to produce drug resistance; (4) it has strong persistence and only affects pathogenic microorganisms or control objects, and will not harm the crops themselves; and (5) it has strong plasticity and can continuously improve performance through molecular biological means, to enhance the effect. Biological pesticides have broad market space and great development prospects, which meet the requirements of my country's agricultural development under the new situation. The integrated agricultural pest control project is an important issue that needs to faced, and biological pesticides will play an important role in it. substitute role.

Rice diseases (such as rice blast, rice false smut, etc.) are important diseases in the process of rice production. At present, the effective way to prevent and control rice blast is to select disease-resistant varieties. However, because the adaptability of varieties requires correct cultivation according to local conditions, it is helpful to establish a good defense concept and take scientific control measures.

Rice disease is one of the three major diseases in rice production. It occurs in different degrees in rice production areas in China. According to the damage part of rice at the time of the onset, rice blast is divided into seedling blast, leaf blast, leaf pillow blast, knot blast, ear-neck blast, grain blast, etc. Among them, ear-neck blast has the most serious impact on yield, which can cause white ears of rice, reduce yield by 40% to 50%, or even stop production. The main ways to prevent and control rice blast are usually: the most effective way is to plant disease-resistant varieties, which is the basis for control, such as glutinous rice, japonica rice with high sensitivity to rice blast, which is difficult and expensive to control; at the same time, due to the adaptability of varieties, it is required to be correct according to local conditions. Therefore, it is the most critical to establish a good defense concept and take scientific control measures. The second is to carry out chemical pesticide control in a timely manner, especially for panicle neck blast with severe yield reduction, it is necessary to carry out two applications of chemical prevention before breaking and at full heading stage. Once the rice variety is selected, chemical control is the second way to prevent rice blast. Line of defense is also the most important control method for rice blast in China.

There are as many as 1,000 pharmaceutical certificates for rice blast, including 620 single doses and more than 300 compound doses. There are still more than 2,000 pesticide production enterprises, and repeated registration of pesticides is common. The pesticides involved in these single doses of rice blast are effective. There are not many available active ingredient options; the total number is not more than 30, with only four that are commercially used to control rice blast. With the improvement of the living environment in rural areas and the requirements of food safety, this kind of agent has been rarely used. Further, the production cost of existing pesticides increases the production cost of the original drug, which leads to the poor cost performance of the product and the loss of market competitiveness. Therefore, the development of environmentally friendly, healthy, low-carbon impact, and/or low-cost pesticides is of great significance to the stability and increase of economic crops and food security.

### SUMMARY OF THE DISCLOSURE

The present disclosure describes methods of making and using Mannosylerythritol lipids (MELs)-containing biological pesticide compositions, based on the fermentative conversion of plant material (e.g., containing component(s) of lignocellulosic hydrolysate or starch hydrolysate and at least one oil/lipid composition). The fermentation is carried out using one or more selected microorganism(s), such as fungi, for instance species of Pseudozyma, Ustilaginales, Moesziomyces, etc. The resultant preparations, which can optionally be extracted and/or otherwise processed to concentrate the produced MEL component(s), can be used to protect plants, such as crop plants, from infection or production loss caused by various infectious microorganisms, including infectious fungi.

One embodiment provides a composition including mannosylerythritol lipid (MEL), produced by fungal fermentation in a growth medium including: at least one carbon source that includes an oil, a yeast extract, sodium nitrite, potassium dihydrogen phosphate, and magnesium sulfate heptahydrate. In examples of the composition, the carbon source includes lignocellulose and its hydrolysate, vegetable oil, and glucose. In embodiments, the growth medium further includes calcium chloride dihydrate, ferrous sulfate heptahydrate, and manganese sulfate.

By way of example, the growth medium may include: 7% to 20% by weight of the carbon source; 0.5% to 2% by weight of sodium nitrate; 0.1% to 0.3% by weight of yeast extract; 0.3% to 0.7% by weight of potassium dihydrogen phosphate; 0.03% to 0.07% by weight of magnesium sulfate heptahydrate; 0.005% to 0.015% by weight of calcium chloride dihydrate; 0.005% to 0.015% by weight of ferrous sulfate heptahydrate; and 0.0005% to 0.0015% by weight of manganese sulfate.

By way of example, the growth medium may include: 15% by weight of the carbon source; 1% by weight of sodium nitrate; 0.2% by weight of yeast extract; 0.5% by weight of potassium dihydrogen phosphate; 0.05% by weight of magnesium sulfate heptahydrate; 0.01% by weight of calcium chloride dihydrate; 0.01% by weight of ferrous sulfate heptahydrate; and 0.001% by weight of manganese sulfate. By way of example, the growth medium may include: 10% by weight of the carbon source; 0.5% by weight of sodium nitrate; 0.3% by weight of yeast extract; 0.25% by weight of potassium dihydrogen phosphate; 0.05% by weight of magnesium sulfate heptahydrate; 0.005% by weight of calcium chloride dihydrate; 0.005% by weight of ferrous sulfate heptahydrate; and 0.002% by weight of manganese sulfate. By way of example, the growth medium may include: 9% by weight of the carbon source; 0.5% by weight of sodium nitrate; 0.3% by weight of yeast extract; 0.25% by weight of potassium dihydrogen phosphate; 0.05% by weight of magnesium sulfate heptahydrate; 0.005% by weight of calcium chloride dihydrate; 0.005% by weight of ferrous sulfate heptahydrate; and 0.002% by weight of manganese sulfate. By way of example, the growth medium may include: 12% by weight of the carbon source; 0.4% by weight of sodium nitrate; 0.3% by weight of yeast extract; 0.25% by weight of potassium dihydrogen phosphate; 0.05% by weight of magnesium sulfate heptahydrate; 0.005% by weight of calcium chloride dihydrate; 0.005% by weight of ferrous sulfate heptahydrate; and 0.002% by weight of manganese sulfate.

In any of the provided composition embodiments 1-8, the fungal microbe that is cultured is one that is recognized for use in producing a MEL; for instance, such microbes include organisms in the genus Moesziomyces (Pseudozyma) and the genus Ustilago.

In a specific composition embodiment, fermentation is carried out with a Pseudozyma fungus in a growth medium including: 15% by weight of carbon source including lignocellulose and its hydrolysate, vegetable oil, and glucose, wherein the mass ratio of the lignocellulose and hydrolysate:vegetable oil:glucose is 1:6:3; 1% by weight of sodium nitrate; 0.2% by weight of yeast extract; 0.5% by weight of potassium dihydrogen phosphate; 0.05% by weight of magnesium sulfate heptahydrate; 0.01% by weight of calcium chloride dihydrate; 0.01% by weight of ferrous sulfate heptahydrate; and 0.001% by weight of manganese sulfate.

In a specific composition embodiment, fermentation is carried out with a Pseudozyma fungus in a growth medium including: 10% by weight of carbon source including lignocellulose and its hydrolysate, vegetable oil, and glucose, wherein the mass ratio of the lignocellulose and hydrolysate:vegetable oil:glucose is 1:7:2; 0.5% by weight of sodium nitrate; 0.3% by weight of yeast extract; 0.25% by weight of potassium dihydrogen phosphate; 0.05% by weight of magnesium sulfate heptahydrate; 0.005% by weight of calcium chloride dihydrate; 0.005% by weight of ferrous sulfate heptahydrate; and 0.002% by weight of manganese sulfate.

In a specific composition embodiment, fermentation is carried out with a Pseudozyma fungus in a growth medium including: 9% by weight of the carbon source including lignocellulose and its hydrolysate, vegetable oil, and glucose, wherein the mass ratio of the lignocellulose and hydrolysate:vegetable oil:glucose is 0.3:0.8:1.7; 0.5% by weight of sodium nitrate; 0.3% by weight of yeast extract; 0.25% by weight of potassium dihydrogen phosphate; 0.05% by weight of magnesium sulfate heptahydrate; 0.005% by weight of calcium chloride dihydrate; 0.005% by weight of ferrous sulfate heptahydrate; and 0.002% by weight of manganese sulfate.

In a specific composition embodiment, fermentation is carried out with a Pseudozyma fungus in a growth medium including: 12% by weight of the carbon source including lignocellulose and its hydrolysate, vegetable oil, and glucose, wherein the mass ratio of the lignocellulose and hydrolysate:vegetable oil:glucose is 0.3:9.2:0.4; 0.4% by weight of sodium nitrate; 0.3% by weight of yeast extract; 0.25% by weight of potassium dihydrogen phosphate; 0.05% by weight of magnesium sulfate heptahydrate; 0.005% by weight of calcium chloride dihydrate; 0.005% by weight of ferrous sulfate heptahydrate; and 0.002% by weight of manganese sulfate.

Also provided are biological pesticides, and biological pesticide compositions, that include the composition of any of the provided embodiments.

Another embodiment is a process for preparing a mannosylerythritol lipid (MEL) composition made by microbial fermentation, which process includes: inoculating a first growth medium including at least one carbon source, sodium nitrite, yeast extract, potassium dihydrogen phosphate, and magnesium sulfate heptahydrate with a seed liquid; wherein the seed liquid is made by inoculating a second growth medium including yeast extract, peptone, glucose, maltose and sodium chloride and water with a microbe and culturing said inoculated second growth medium; culturing said inoculated first growth medium to produce a fermentation broth; temperature sterilizing the fermentation broth to obtain a sterilized fermentation broth; filtering the sterilized fermentation broth, to produce a filtrate; adjusting the pH of the filtrate to an acidic pH to obtain a microbe separation liquid wherein the microbe separation liquid is miscible in an least one organic solvent (e.g., ethyl acetate or chloroform); and extracting the microbe separation liquid with the at least one organic solvent, to produce the MEL composition.

In examples of this process embodiment, the first growth medium further includes calcium chloride dihydrate, ferrous sulfate heptahydrate, and manganese sulfate.

In representative process embodiments, the first growth medium includes: 7% to 20% by weight of the carbon source; 0.5% to 2% by weight of sodium nitrate; 0.1% to 0.3% by weight of yeast extract; 0.3% to 0.7% by weight of potassium dihydrogen phosphate; 0.03% to 0.07% by weight of magnesium sulfate heptahydrate; 0.005% to 0.015% by weight of calcium chloride dihydrate; 0.005% to 0.015% by weight of ferrous sulfate heptahydrate; and 0.0005% to 0.0015% by weight of manganese sulfate.

In representative process embodiments, the first growth medium includes: 15% by weight of the carbon source; 1% by weight of sodium nitrate; 0.2% by weight of yeast extract; 0.5% by weight of potassium dihydrogen phosphate; 0.05% by weight of magnesium sulfate heptahydrate; 0.01% by weight of calcium chloride dihydrate; 0.01% by weight of ferrous sulfate heptahydrate; and 0.001% by weight of manganese sulfate.

In representative process embodiments, the first growth medium includes: 10% by weight of the carbon source; 0.5% by weight of sodium nitrate; 0.3% by weight of yeast extract; 0.25% by weight of potassium dihydrogen phosphate; 0.05% by weight of magnesium sulfate heptahydrate; 0.005% by weight of calcium chloride dihydrate; 0.005% by weight of ferrous sulfate heptahydrate; and 0.002% by weight of manganese sulfate.

In representative process embodiments, the first growth medium includes: 9% by weight of the carbon source; 0.5% by weight of sodium nitrate; 0.3% by weight of yeast extract; 0.25% by weight of potassium dihydrogen phosphate; 0.05% by weight of magnesium sulfate heptahydrate; 0.005% by weight of calcium chloride dihydrate; 0.005% by weight of ferrous sulfate heptahydrate; and 0.002% by weight of manganese sulfate.

In representative process embodiments, the first growth medium includes: 12% by weight of the carbon source; 0.4% by weight of sodium nitrate; 0.3% by weight of yeast extract; 0.25% by weight of potassium dihydrogen phosphate; 0.05% by weight of magnesium sulfate heptahydrate; 0.005% by weight of calcium chloride dihydrate; 0.005% by weight of ferrous sulfate heptahydrate; and 0.002% by weight of manganese sulfate.

In various process embodiments, the second growth medium includes: 0.2% to 0.4% by weight of yeast extract; 0.4% to 0.8% by weight of peptone; 0.2% to 0.6% by weight of glucose; 0.05% to 0.15% by weight of maltose; and 0.3% to 0.7% by weight of sodium chloride. In various process embodiments, the second growth medium includes: 0.3% by weight of yeast extract; 0.6% by weight of peptone; 0.4% by weight of glucose; 0.1% by weight of maltose; and 0.5% by weight of sodium chloride. Optionally, in any process embodiment, the remainder of a growth medium is water.

Various process embodiments further include maintaining the inoculated first growth medium at a temperature of from 22° C. to 32° C., or from 25° C. to 28° C. Optionally, the process embodiments further include stirring the inoculated first growth medium. Optionally, in process embodiments the inoculated first growth medium is cultured for from 5 days to 15 days, for instance for 10 days.

Various process embodiments further include maintaining the second inoculated growth medium at a temperature of from 25° C. to 35° C., for instance at a temperature of 30° C. Optionally, in process embodiments the inoculated first growth medium is cultured for from 1 day to 3 days.

In various process embodiments, temperature sterilization is carried out at: a temperature of from 60° C. to 140° C.; or a temperature of from 80° C. to 120° C. Optionally, the sterilization temperature is maintained for a period for from 1 hour to 3 hours, for instance for 2 hours.

Optionally, the process embodiments further include isolating the organic phase from the extraction of the microbe separation liquid, and optionally removing the organic solvent from the organic phase to provide a concentrated microbe separation liquid.

Also provided are MEL compositions made by any of the process embodiments described herein, as well as biological pesticides that include such a MEL.

Yet another embodiment is a method of preventing or treating a crop disease caused by a crop pathogen, including contacting the crop plant or a part thereof infected by or possibly exposed to the crop pathogen (or containing a growth medium in which the plant is growing or may grow) with a therapeutically effective amount of the described biological pesticide(s). By way of example, the crop disease includes rice blast, Sorghum hard smut, rice smut, corn leaf blight, and other fungal diseases. More generally, the disease may be a rice disease (such as rice blast, or rice false smut disease), or another disease listed herein or recognized in the art.

In examples of these method embodiments, the crop is a monocot plant, such as rice, wheat, corn, or sorghum; or another commercially relevant crop plant. Additional plants, including crop plants, are known and are discussed herein.

### BRIEF DESCRIPTION OF THE DRAWINGS

FIG. 1 shows a series of petri dishes illustrating the inhibitory effect of a described biopesticide preparation (see Example 1), at the indicated dilutions, on rice blast fungus.

FIG. 2 shows a series of petri dishes illustrating the inhibitory effect of a described biopesticide preparation (see Example 2), at the indicated dilutions, on green smut.

FIG. 3 shows a series of petri dishes illustrating the inhibitory effect of a described biopesticide preparation (see Example 3), at the indicated dilutions, on rice fever fungus.

FIG. 4 shows a pair of petri dishes illustrating the inhibitory effect of a described biopesticide preparation (see Example 4), at the indicated dilution, on Bipolaris maydis.

FIG. 5 shows a pair of petri dishes illustrating the inhibitory effect of a described biopesticide preparation (see Example 5), at the indicated dilution, on Rhizoctonia solani.

### DETAILED DESCRIPTION

The present disclosure describes methods of making and using Mannosylerythritol lipids (MELs)-containing biological pesticide compositions, based on the fermentative conversion of plants, plant-derived materials, or derivatives thereof—including one or more oil(s) along with at least one other non-oil/lipid-based carbon source. The fermentation is carried out using microorganism(s), such as fungi, for instance species of Pseudozyma, Ustilaginales, Moesziomyces, etc. The resultant biological pesticide preparations can be used to protect plants, such as crop plants, from infection or production loss caused by various infectious microorganisms. For fermentation strains, bioactive components obtained by transformation, biochemical reaction, extraction and other processes of plants, their derivatives or plant-derived materials. The invention provides the synthesis, separation, concentration and application of the biological pesticide in the prevention of crop pathology.

MELs are well known in the art. See, for instance, Beck & Zibek (Front. Bioeng. Biotechnol. 8, 2020, doi.org/10.3389/fbioe.2020.555280); Morita et al. (J Oleo Sci. 64(2):133-141, 2015, doi:10.5650/jos.ess14185); Ceresa et al. (Curr. Microbiol. 77(8):1373-1380, 2020, doi: 10.1007/s00284-020-01927-2); and de Andrade et al. (Separ. Purif. Tech. 180:157-167, 2027).

Representative Structure of Mannosylerythritol Lipids (Formula I):

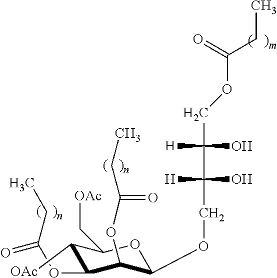

Embodiments of the provided methods for conversion of plant-based materials (such as lignocellulose-containing or derived, or starch-containing preparations), by fermentation, into biopesticide compositions mainly include the following steps:

(1) Seed Preparation: Colonies are picked from pure microbe cultures, such as solid culture plates, of the selected fermentation microorganism. In various embodiments, the microbe is Pseudozyma (Moesziomyces), or another Ustilaginales family member, or any other microbe known to produce MEL(s) or engineered to do so. The organism is inoculated into growth medium, such as the following medium: yeast powder 0.3% (e.g., yeast extract powder, such as is typically used for growth medium; or bi-products from production of fermentation yeast; usually a preparation from yeast fermentation largely lacking water-insoluble components), peptone 0.6%, glucose 0.4%, maltose 0.1%, sodium chloride 0.5%, where the rest of the volume is water, transferred to a constant temperature culture of 30° C. After being cultured for 1-3 days, this provides the seed liquid culture (starter inoculum) for each strain.

(2) Synthesis: Inoculate the inoculum of 1%-10% (e.g., 90 ml medium inoculated with 10 mls of seed culture is 10%), transfer the seed liquid of the above-mentioned fermentation microorganism(s) into the prepared culture medium, using lignocellulose and its hydrolysate (which generally contains the monosaccharaide, xylose, and other components; different biomass hydrolysates contains the various percentages; see e.g., Jonsson et al., Biotechnology for Biofuels, 6:16, 2013), vegetable oil (such as almond oil, coconut oil, corn oil, cottonseed oil, grapeseed oil, hazelnut oil, linseed (flaxseed) oil, olive oil, palm (kernel) oil, peanut oil, rapeseed (canola) oil, rice bran oil, safflower oil, sesame oil, soybean oil, sunflower oil, walnut oil, and mixtures of two or more thereof; recaptured oils, such as used culinary oils, as well as industrial oils are also contemplated), glucose, sucrose, starch and its hydrolysate, maltose, etc. as the main carbon sources, with culture aeration of 0.01-1.0 vvm. The temperature is controlled at 20-40° C., and the stirring intensity is 100-300 rpm. The medium is: carbon source 1-15%, sodium nitrate 0.1-3%, yeast powder 0.01-2%, potassium dihydrogen phosphate 0.1-4.5%, magnesium sulfate heptahydrate 0.01-0.4%, calcium chloride dihydrate 0.01-0.1%, ferrous sulfate heptahydrate 0.01-0.03%, manganese sulfate 0.001-0.01%. After culturing for 4-15 days, the fermentation broth of each microbe was obtained.

(3) Separation: The fermentation broth obtained in the above-mentioned fermentation process is sterilized, for instance by high temperature sterilization at 80-120° C. for 2 hours. The sterilized fermentation broth is filtered to remove bacterial cells; filtration can be carried out using known methods, such as filtration membranes or filter bags, etc. The residual raw-material (including oil) is removed from the cell-free supernatant by the high speed centrifugation; the liquid obtained after centrifugation is harvested, adjusted to a pH of 3 or lower, and chilled at a temperature of 4-10° C. at 24 hours.

(4) Concentration: Organic solvent extraction can be used, such as ethyl acetate, chloroform, etc., and then the organic phase is collected. The liquid obtained by the above separation method is fully mixed with solvents of different volumes, and then left to stand for separation. It is then vacuum concentrated 10-15 times at 40-60° C., and the solvent is collected for recycling. The process can also be extracted through organic or ceramic membranes, followed by vacuum concentration at 40-60° C.

(5) Application test: The biopesticide obtained using the above process is used for crop disease control test. Prepare a solid medium, dilute the biological pesticide to a certain number, spread it evenly on the surface of the solid medium, and then spot the crop germs in the center of the fixed plate, such as rice blast fungus, rice koji fungus, etc. Finally, it was placed in an incubator to observe its inhibitory function.

(6) Structural analysis of main components: the extract in step 4 may be subjected to structural analysis by means of high performance liquid chromatography, mass spectrometry, gas chromatography, NMR and the like.

Though exemplary organisms are described herein, there are other member of the genus Moesziomyces (Pseudozyma) and the genus Ustilago that can be used to produce MELs. See, for instance, Beck & Zibek (Front. Bioeng. Biotechnol. 8, 2020, doi.org/10.3389/fbioe.2020.555280) and references cited therein; Morita et al. (J Oleo Sci. 64(2):133-141, 2015, doi:10.5650/jos.ess14185); Ceresa et al. (Curr. Microbiol. 77(8):1373-1380, 2020, doi: 10.1007/s00284-020-01927-2); and de Andrade et al. (Separ. Purif. Tech. 180:157-167, 2027); Ramdass & Rampersad (BMC Microbiol. 22, Art. 43, 2022); Becker et al. (Metabolic Eng. Comm. 12:e00164, 2021, doi.org/10.1016/j.mec.2021.e00165); and de Silva et al. (Bioprocess Biosys. Eng. 44:2003-2034, 2021, doi.org/10.1007/s00449-021-02597-5).

### Representative Terms

Active agent as used herein refers to a chemical or compound that has a particular biological activity. Active agents may include chemicals or compounds that have acaricidal activity, bactericidal activity, fungicidal activity, herbicidal activity, insecticidal activity, larvicidal activity, nematocidal activity, miticidal activity, molluscicidal activity, piscicidal activity, rodenticidal activity, slimicidal activity, or are a fertilizer, a hormone and/or other growth regulator. Additional active ingredients are listed herein. In addition, active agents may include chemicals or compounds that support or enhance plant growth. Active agents may also be referred to as active ingredients. The MEL-containing biological pesticides described herein are an example active agent. Any contemplated agricultural composition may contain more than one active agent, where at least one of the active agent(s) is a MEL-containing biological pesticide described herein and/or produced by a method described herein.

Adjuvants as used herein refers to an ingredient that aids or modifies the biological activity and/or physical properties of a formulation.

The use of adjuvants with agricultural chemicals generally falls into four categories: (1) activator adjuvants which generally enhance performance of a formulation, (2) spray modifier adjuvants which generally affect the application performance of spray solutions (e.g. drift retardants, stickers, evaporation aids), (3) utility modifiers which generally minimize handling and improve application (e.g., anti-foam agents), and (4) utility products that minimize application problems (e.g. foam markers and tank cleaners).

Agriculturally acceptable adjuvant as used herein refers to a substance that enhances the performance of an active agent in a composition that is used to influence (that is, inhibit or enhance, depending on circumstances) the growth or cultivation of plants and/or plant parts.

Agrochemical as used herein refers to any chemical substance used to help manage an agricultural ecosystem, such as, for example, a hormone or other growth regulator, a pesticide (such as an herbicide, insecticide, fungicide, nematicide, miticide, larvicide, molluscicide, and so forth), a fertilizer, a soil conditioner, a liming agent, an acidifying agent, or any other growth agent.

Ambient temperature as used herein refers to the temperature at a location or in a room, or the temperature which surrounds an object under discussion. This term is equivalent to “room temperature” (rt). By way of example, room temperature may be between 65° F. and 78° F. (about 18.3° C. to 25.5° C.); or between 68° F. and 72° F. (about 20° C. to 22.2° C.).

Aqueous dispersion as used herein refers to a water-based formulation in which a compound has been dispersed. In specific embodiments, or in combination with any of the mentioned embodiments, an aqueous dispersion of a sulfopolyester is a formulation in which a sulfopolyester compound has been dispersed in water. An aqueous dispersion formulation can have a continuous phase of water in contrast to a continuous phase of organic solvent.

Concentrate formulation (a.k.a., formulate concentrate) as used herein refers to a formulation that contains at least one active agrochemical compound at a level at least two-times the level used in an as-applied formulation, or at a level higher than the level at which the active ingredient is in a ready to use (RTU) formulation. Thus, a concentrate formulation is expected or intended to be diluted (for instance, with water or another acceptable carrier or diluent) before use or application. In representative embodiments, or in combination with any of the mentioned embodiments, a concentrate formulation includes at least one active ingredient at a level that is at least twice as concentrated as that ingredient would be used in an as-applied or RTU formulation.

Contact angle as used herein refers to a profile measurement of a drop of water in contact with a solid surface; the flatter a droplet, the lower the contact angle reading. In specific embodiments, or in combination with any of the mentioned embodiments, adjuvants (e.g. surfactants), can reduce surface tension, spreading out a water droplet and decreasing the contact angle.

Control formulation as used herein is a formulation that contains the same ingredients as a reference formulation, but without any MEL-containing biological pesticide described herein. Optionally, the control formulation may include fungicide(s) in place of the MEL-containing biological pesticide described herein, such as art-recognized fungicide(s) that are believed to perform function(s) similar to the function(s) for which the MEL-containing biological pesticide described herein is included in one embodiment or in combination with any of the mentioned embodiments of the reference formulation.

Diluent as used herein refers to a gas, liquid, or solid used to reduce the concentration of an active ingredient in the formulation or application of an agrochemical composition.

Drift as used herein refers to the airborne movement of a compound from an area of application to any unintended (e.g., off-target) site. Drift can happen during agrochemical application, for instance when droplets or particles travel away from the target site. Drift can also happen after the application, when some chemicals become vapors that can move off of the application site.

Drift includes everything that comes off of or out of the target (plant, plant part, growth medium, etc.). Many phenomena contribute to drift, such as for instance evaporation or sublimation, as well as off-target spray deposition. These are the two predominant forms of drift that are often considered in agricultural embodiments; both are important to control impacts on neighboring fields. The two main forms are: Particle or Droplet drift (movement of spray droplets produced at the time of application), which can be influenced by rheology modifiers that affect the size of droplets coming out of the sprayer; and Vapor drift (movement of fumes/vapors after a volatile formulation is applied), which can be influenced by modifying the volatility of the formulation as well as modifying the circumstances under which a compound is applied.

Drift control as used herein refers to the act or effect of measurably reducing or preventing drift. In representative embodiments, drift control includes a statistically significant reduction in drift of a detectable compound, for instance in a comparison between formulations that have one component different in presence or amount. Drift control agents are chemical agents that reduce one or more of: wind drift experienced when spraying a tank mix composition, or vapor drift. Example drift control agents increase droplet size and/or reduce the proportion of driftable fines (droplets of less than 150 microns) in a formulation, for instance by increasing viscosity of the formulation. There are art-recognized standard methods to measure drift; see, for instance, US Patent Publication 20160015033 A1.

Effective amount as used herein refers to an amount sufficient to cause a beneficial and/or desired result. For example, an active ingredient can be present in a formulation at an amount effective to provide the desired effect linked to that active ingredient, such as a pesticide effect, a fungicidal effect, or any other agrochemical effect. The amount of any active or other ingredient that is effective for its desired use is usually influenced by what ingredient is being used, the context in which it is used (for instance, other components in a formulation), the method or manner in which the composition containing the ingredient is being used, and so forth. An effective amount for any particular ingredient and in various contexts can be determined using art-recognized methods.

Emulsion as used herein refers to a mixture that results when one liquid is added to another and is mixed with it but does not dissolve into it, creating a homogeneous dispersion of liquid droplets dispersed in the continuous phase. An emulsion is a made by combining two liquids that normally do not mix. The process of turning a liquid mixture into an emulsion is called emulsification.

Flowable concentrate as used herein refers to a suspension of one or more solid active ingredients (at a level at least two-times the level used in an as-applied or RTU formulation) in water.

Growth medium as used herein refers to any natural or artificial solid, semi-solid or liquid that is suitable for germination, rooting, and/or propagation of plants. Examples of growth media include peat moss, vermiculite, perlite, wood bark, coir, sawdust, certain types of fly ash, pumice, plastic particles, glass wool, rock wool, and certain polymer-based foams. These are commonly used either alone or in various combinations with each other and/or natural soil (with or without soil amendments). Suitable soil amendments include ground natural minerals, such as kaolins, clays, chalk, and talc; ground synthetic minerals, such as silicates and highly dispersed silica; anionic or non-ionic emulsifiers; surfactants, such as alkali metal salts lignosulfate and naphthalene sulfonic acid; and dispersing agents, such as methylcellulose. Natural soil is also contemplated as a growth medium herein, including in situ soil in fields. The term growth medium also specifically includes liquid media used for hydroponic plant growth, as well as growing support materials/substrates used in conjunction with hydroponic processes.

Inert Ingredient or Component as used herein refers to any substance other than an active ingredient (such as an agrochemical active ingredient) that is intentionally included in a formulation. Non-limiting examples of inert ingredients include emulsifiers, solvents, carriers, sticker agents, surfactants, drift control agents, drought control agents, fragrances, dyes and adjuvants with spreader activity, with rain fastness activity, and so forth.

Lipophilic compound as used herein is a compound tending to combine with or dissolve in lipids or fats. In general, lipophilic compounds have solubility in water that is in the “sparingly soluble” range, or lower. For compounds that are “sparingly soluble in water,” the quantity of water needed to dissolve one gram of the compound will be in the range beginning at 30 mL and ending at 100 mL or higher. Compounds having solubility lower than “sparingly soluble” in water will require greater volumes of water to dissolve the compounds.

Loadings as used herein refers to the amount of a material in a given volume. For agricultural formulations, loading(s) often refers to the amount of active ingredient in the formulation, represented as a g/liter percentage.

Oil Dispersion (OD) as used herein refers to a system in which distributed particles (liquid or solid) of a material are uniformly dispersed in a continuous phase of an oil. Water sensitive active agents are usually formulated as solid dry formulations, as they are hydrolytically unstable active agents. The OD enables water sensitive active agents to be formulated as liquid formulations. In an OD, the water sensitive solid or liquid particles are homogeneously suspended in the oil phase. The oil in the formulation have the added features of foliar absorption enhancement and spray retention on the leaves by hydrophobic affinity. As oil dispersions can optionally be water free, there is no need to add biocides as preservatives, which is an advantage to using oil dispersions.

Oil-in-Water emulsion as used herein refers to a mixture wherein oil is dispersed as extremely fine droplets in a continuous phase of water. Optionally, one or more active ingredient(s) may also be contained in an oil-in-water emulsion; depending on the active ingredient, it may be contained in the oil phase, the water phase, or both.

Pest as used herein is any organism (including microorganisms) in a circumstance that makes the presence of the pest undesirable. It is recognized that a pest in exemplary instances is a plant (e.g., a weed), a microorganism (such as a fungus, bacteria, nematode, and so forth), an insect (including any phase or life cycle of an insect, such as eggs, larvae, or adult insects), a mollusk (such as a slug or snail), or a larger animal (such as a rodent, bird, fish, and so forth).

Pesticide as used herein include any substance or mixture of substances intended for preventing, destroying, repelling, or mitigating any unwanted pest, wherein a pest is any organism that may have an impact on a crop. There are many subcategories of pesticides, which include: insecticides, herbicides, rodenticides, bactericides, fungicides, larvicides, miticides, molluscicides, nematicides, and so forth.

Phase as used herein refers to a physically distinctive form of matter. While they are canonically thought of in the form of solids, liquids, gases, or plasmas, there are other phases that are important for mixtures. For example, in emulsions there are two phases, a continuous phase and a dispersed phase that occupies disconnected regions of space. A dispersed phase can coalesce and yet remain as a dispersed phase, until and unless the coalescing forms a continuous connection throughout a given volume, at which point it becomes a continuous phase. A dispersed phase can be discrete droplets of liquid, solids, or gas bubbles in a continuous phase.

Phytotoxicity as used herein refers to any form of plant injury. Phytotoxicity can cause one or more of the following to the plant: leaf tip or edge burn, overall yellowing, stunting, small leaf size, leaf curling, cupping and other distortions, dark green color (typical of triazole fungicides), speckling, delays in flowering, delays in rooting, delays or reductions in seed or fruit development, or plant death. A substance, compound, composition, or formulation that is “substantially non-phytotoxic” will not produce any of the aforementioned adverse effects when applied to a plant.

In testing for phytotoxicity, a substance, compound, composition or formulation is applied to the plant of interest and the plant is visually observed for a period of time, such as an hour, a day, a week, multiple weeks, a month, or an entire growing season. Measurement of phytotoxicity can be done visually (for instance, leaf impacts or total plant health observation) or quantitatively (for instance, amount of fruit or seed produced). If the substance, compound, composition or formulation is substantially non-phytotoxic, there will be no statistically relevant difference in appearance, or production, relative to a non-treated plant.

The term “plant” is used herein in its broadest sense. It refers to a whole plant including any root structures, vascular tissues, vegetative tissues, and reproductive tissues. A “plant part” includes any portion of a plant. A plant part is any part of a plant, tissue of a plant, or cell of a plant. In particular embodiments, a plant or plant part includes: a whole plant, a seedling, cotyledon, meristematic tissue, ground tissue, vascular tissue, dermal tissue, seed, pod, tiller, sprig, leaf, stomata, root, shoot, stem, flower, fruit, pistil, ovaries, pollen, stamen, phloem, xylem, stolon, plug, bulb, tuber, corm, keikis, bud, and blade. In particular embodiments, a plant part includes a root, a stem, or a leaf. For example, upon harvesting a tree, the tree separated from its roots becomes a plant part. “Leaf” and “leaves” refer to a usually flat, green structure of a plant where photosynthesis and transpiration take place and attached to a stem or branch. “Stem” refers to a main ascending axis of a plant. “Seed” refers to a ripened ovule, including the embryo and a casing.

Plants include any (but are not limited to) species of grass (e.g., turf grass), sedge, rush, ornamental or decorative plants, crop or cereal plants, fodder or forage plants, monocot or dicot plants, fruit plants or vegetable plants, flowers, and trees. In particular embodiments, a plant includes: wheat, soybean, maize (corn), barley, millet, oats, rye, rice, sorghum, palm, coconut, sugar cane, turfgrass, other agriculturally important monocots (including palms, bananas and plantains, ginger and relatives, turmeric and cardamom, vanilla, asparagus, pineapple, sedges and rushes, and alliums (e.g., leeks, onion, shallots, and garlic)), cotton, canola, sunflower, rapeseed (and other oil-producing, seed bearing plants), alfalfa, tomato, sugar beet, almond (and other tree nuts), walnut, apple, cannabis, peanut, strawberry, lettuce, orange (and other citrus, such as lemon, lime, grapefruit and so forth), potato, banana, cassava, mango, guava, olives, peppers, tea, yams, cacao, asparagus, carrot, watermelon (and other melons), cabbage, cucumber (and other cucurbits), and grape.

Preservative as used herein is any chemical that inhibits or suppresses decomposition of a product or formulation, such as an agrochemical formulation.

Rainfastness as the term is used herein is a measure of how well a substance, after application to a surface (such as a leaf surface), resists being washed away by rainfall or irrigation. A formulation is considered rainfast after application when and if it has adequately dried or has been absorbed by plant tissues so that it will still be effective after rainfall or irrigation. The degree of rainfastness of agrochemical formulations is highly variable. The art recognizes methods for determining or measuring rainfastness of a formulation. For instance, other tests may be based on visual determination of an amount of a marker dye residue left on the leaves (or other test application surface) after “rain” or other washing. By way of example, a fluorescent dye or colored dye may be added to the formulation prior to application to the surface. After the formulation is allowed to dry, the amount of dye may be determined visually or with the use of a fluorescence detector or colorimetric detector. Following rain or exposure to water, the leaf or surface can be dried and again evaluated for residual dye. Comparison to a control formulation provides an indication of the effective rainfastness of the modified formulation.

Solvent dispersion as used herein refers to a system in which distributed particles of a material are uniformly dispersed in a continuous phase of a substantially water-immiscible solvent. An oil dispersion is a variety of solvent dispersion.

Sprayability as the term is used herein refers to the ability of a liquid or gel to be driven or dispersed in air as, for example, particles, drops or droplets

Spread as used herein refers to the act of or the ability of a formulation (such as a mixture, dispersion, or emulsion) to extend, distribute, cover, or coat a certain area. In particular embodiments, spread refers more particularly to the act of or the ability of a formulation to overcome at least in part the hydrophobic nature of the surface of a plant or plant part, thus allowing a formulation to attain better contact and/or coverage with the formulation. The ability of a formulation to spread can be measured using standard tests known to those in the art, such as contact angle measurements.

The ability of a liquid composition to spread onto a surface is related to the surface tension of that liquid. Thus, surfactants, detergents, and other compounds that reduce surface tension can be used to increase spread.

Sticker or sticker adjuvant as used herein refers to a compound or ingredient used in an agrochemical formulation that influences the deposition characteristic(s) of the formulation to allow it to “stick” on a surface better than a formulation without that compound or ingredient. A sticker adjuvant provides one or more of: increased surface contact between the formulation and a surface on which it is sprayed; reduced runoff; and/or increased surface penetration. At least some sticker adjuvants exhibit surfactant activity.

Surface tension as used herein refers to the condition that exists at the free surface of a liquid. Surface tension is a measure of the force required to pull a floating ring off the surface of a liquid and is measured in dynes/cm.

Surfactant as used herein refers to a compound that lowers the surface tension (or interfacial tension) between two liquids, between a gas and a liquid, or between a liquid and a solid. Surfactants may act as detergents, wetting agents, emulsifiers, foaming agents, and dispersants. Surfactants may amphoteric, nonionic, and/or anionic. In an agrochemical formulation, surfactants may influence one or more of: emulsification, dispersion of active ingredient(s), spreading, and/or wetting.

Suspension as used herein refers to a heterogeneous mixture that contains solid particles dispersed in a liquid where the solid particles do not completely dissolve in the liquid. The particles may be visible to the naked eye and may eventually settle, although the mixture is only classified as a suspension when and while the particles have not settled out.

Ready to use (RTU) as used herein refers to a formulation that requires no further dilution before application.

Tank mix as used herein refers to two or more chemical pesticides, inert ingredients, components, or formulations, mixed in the spray tank at the time of spray application or immediately before.

Thickener as used herein refers to a material a primary function of which is to increase the viscosity of a fluid.

Volatilization as used herein refers to the process by which a dissolved sample is vaporized or a solid residue is sublimed.

Water Hardness as used herein is a measure of the amount of minerals that are present in water. Hardness is typically expressed in milligrams of dissolved calcium and magnesium carbonate per liter of water; however, other bivalent and trivalent metallic elements may contribute to water hardness.

Water immiscible as used herein refers to a liquid, generally a solvent, that has limited or no significant ability to mix with water or an aqueous phase at ambient conditions. That is, in the absence of a surfactant, a water immiscible solvent mixed with water will form two layers when in spite of possible slight solubility. The term is not intended to be absolute, and it is recognized that hydrophobic liquids (such as oils and other hydrophobic solvents) may in fact be able to mix with water to a limited extent. Thus, in one embodiment or in combination with any of the mentioned embodiments, a water immiscible solvent will be less than 0.1%, less than 0.2%, less than 0.3%, less than 0.4%, less than 0.5%, less than 0.75%, less than 1%, less than 1.25%, less than 1.5%, less than 2%, less than 2.5%, less than 3%, less than 5%, less than 7%, less than 8%, less than 9, or 0.1-10% soluble/mixable with water at ambient conditions. Examples of water immiscible solvents include: mineral oil, vegetable oils, seed oils, methylated seed oils, banana oil, white mineral oil mineral spirits, toluene, benzene, xylene, acetophenone, isopropyl acetate, t-butyl acetate, methyl n-propyl ketone, propyl acetate, methyl isobutyl ketone, isobutyl acetate, n-propyl propionate, butyl acetate, methyl isoamyl ketone, methyl amyl acetate, n-butyl propionate, p-amyl acetate, methyl n-amyl ketone, isobutyl isobutyrate, cyclohexanone, di-isobutyl ketone, n-pentyl propionate, ethyl 3-ethoxy propionate, 2-ethylhexyl acetate, ethylene glycol monobutyl ether, isophorone, 2,2,4-trimethyl-1,3-pentanediol monoisobutyrate, 2,2,4-trimethyl-1,3-pentanediol diisobutyrate, 2-heptanol, or 2-ethyl hexanol.

Water-in-Oil emulsion as used herein refers to a mixture wherein water is dispersed as extremely fine droplets in a continuous phase of oil or other water immiscible solvent. Optionally, one or more active ingredient(s) may also be contained in a water-in-oil emulsion; depending on the active ingredient, it may be contained in the oil phase, the water phase, or both. A water-in-oil emulsion is an example of a water-in-water immiscible solvent emulsion.

### Common Fungal Diseases and Crops Affected

The MEL-containing biological pesticidal compositions provided herein are effective against a wide range of agriculturally relevant fungal organisms.

Fungi constitute the largest number of plant pathogens and are responsible for a range of serious plant diseases. Most vegetable diseases are caused by fungi, which damage plants by killing cells and/or causing plant stress. Sources of fungal infections are infected seed, soil, crop debris, nearby crops, and weeds. Fungi are spread by wind and water splash, and through the movement of contaminated soil, animals, workers, machinery, tools, seedlings and other plant material. They enter plants through natural openings such as stomata, and through wounds such as those caused by pruning, harvesting, hail or other weather damage, insects, other diseases, and mechanical damage.

Some of the fungi are responsible for foliar diseases—Downy mildews; Powdery mildews; and White blister are some of the highly prevalent foliar diseases. Other fungi—Clubroot; Pythium species; Fusarium species; Rhizoctonia species; Sclerotinia and Sclerotium species—are soilborne diseases.

Many fungal diseases occur on a wide range of vegetables. These diseases include Anthracnose, Botrytis rots, Downy mildews, Fusarium rots, Powdery mildews, Rusts, Rhizoctonia rots, Sclerotinia rots, Sclerotium rots, and so forth. Others are specific to a particular crop group, e.g., Clubroot (Plasmodiophora brassicae) in brassicas, Leaf blight (Alternaria dauci) in carrots, and Red root complex in beans.

Some examples of common fungal diseases of vegetable crops are provided in the following table (source: ausveg.com.au/biosecurity-agrichemical/crop-protection/overview-pests-diseases-disorders/fungal-diseases/):

| | Representative Crops | |
|---|---|---|
| Fungal Disease | Affected | Symptoms |
| White blister/White rust | Brassicas (including Asian | White blisters and swellings on |
| (Albugo candida) | leafy brassicas) | leaves and heads of affected plants; |
| | | blisters consist of masses of white |
| | | dust-like spores; up to 100% losses |
| | | have been reported. |
| Downy mildews | Wide host range including | Symptoms usually begin with |
| (individual species | onions; peas; lettuce; | yellowish leaf spots which then turn |
| damage particular crop | celery; spinach; kale; herbs; | brown; downy growth appears on |
| families) | cucurbits; brassicas; Asian | underside of leaves. |
| | leafy brassicas | |
| Powdery mildews (some | Wide host range and very | Small, white, powdery patches on |
| species are restricted to | common, especially in | most above-ground surfaces; |
| particular crops or crop | greenhouse crops: | usually observed first on undersides |
| families) | cucumber; melons; | of leaves but eventually cover both |
| | pumpkin; zucchini; parsnip; | surfaces; affected leaves become |
| | beetroot; potato; herbs; | yellow, then brown and papery and |
| | peas; bitter melon; tomato; | die. |
| | capsicum; Brussels sprouts; | |
| | cabbage; swedes | |
| Clubroot | Brassicas (including Asian | Plants are yellow and stunted and |
| (Plasmodiophora | leafy brassicas). | may wilt in hotter parts of the day; |
| brassicae) | | large malformed ‘clubbed’ roots |
| | | which prevent the uptake of water |
| | | and nutrients, reducing the potential |
| | | yield of the crop. |
| Pythium species | Many vegetable crops in | May kill seedlings, which die before |
| | including cucurbits; | they emerge or soon after |
| | brassicas; lettuce | emergence; plant collapse. |
| Sclerotinia rots (S. | Most vegetable crops | Water-soaked rotting of stems, |
| sclerotiorum and S. | | leaves, and sometimes fruit; |
| minor) | | followed by a fluffy, white and |
| | | cottony fungal growth which contain |
| | | hard black pebble-like sclerotia. |
| Sclerotium rots | S. rolfsii-Wide host range | S. rolfsii-Lower stem and root rots; |
| (Sclerotium rolfsii and | including: beans; beets; | coarse threads of white fungal |
| S. cepivorum) | carrot; potato; tomato; | growth surround the diseased |
| | capsicum; cucurbits. S. | areas; small brown fungal resting |
| | cepivorum-only affects | bodies. S. cepivorum-Yellowing |
| | onions, garlic and related | and wilting; fluffy fungal growth |
| | Alliums (shallots; spring | containing black sclerotia forms at |
| | onions; leeks) | the bases of bulbs. |
| Fusarium wilts and rots | Wide host range including: | Causes severe root and crown rots |
| (Various Fusarium | brassicas; carrots; | or wilt diseases by attacking roots |
| species including F. | cucurbits; onions; spring | and basal stems; cucurbit fruit and |
| solani and F. | onions; potato; tomato; | potato tubers can be affected in |
| oxysporum) | herbs; peas; beans | storage. |
| Botrytis rots-for | Celery; lettuce; beans; | Softening of plant tissues in the |
| example Grey mold | brassicas; cucumber; | presence of grey fungal growth. |
| (Botrytis cinerea) | capsicum; tomato | |
| Anthracnose | Wide range of crops | Typical symptoms begin with |
| (Colletotrichum | including: lettuce; celery; | sunken and water-soaked spots |
| spp. except for in lettuce- | beans; cucurbits; tomato, | appearing on leaves, stems and/or |
| Microdochium | capsicum; potato; globe | fruit. |
| panattonianum) | artichoke | |
| Rhizoctonia rots | Wide host range including: | Range of symptoms depending on |
| (Rhizoctonia solani)- | lettuce; potato; brassicas; | the crop being grown but can affect |
| range of common | beans; peas; beets; carrots; | roots, leaves, stems, tubers and |
| names, e.g., Bottom rot | capsicum; tomato; cucurbits | fruit; plants wilt and may collapse |
| (lettuce) and Wire stem | | and die. |
| (Brassicas) | | |
| Damping-off (Pythium, | Many vegetable crops | Young seedlings have necrotic |
| Rhizoctonia, | including: leafy vegetables; | stems or roots; seedlings die or |
| Phytophthora, Fusarium | brassicas; carrots; beetroot; | show a reduction in growth. |
| or Aphanomyces) | cucurbits, eggplant; tomato; | |
| | coriander; spring onions; | |
| | beans | |
| Cavity spot (Pythium | Carrots | Cavity spots are small elliptical |
| sulcatum) | | lesions often surrounded by a yellow |
| | | halo. |
| Tuber diseases | Potato and sweet potato | Potato tubers may be infected with |
| | | superficial skin diseases, such as |
| | | common scabs, powdery scab, and |
| | | Rhizoctonia. Sweet potatoes may |
| | | be infected by scurf. |
| Rusts | Sweet corn; beans; onions; | Small, red or reddish-brown |
| (several species, | spring onions; beets; celery; | pustules that form on the underside |
| e.g., Puccinia sorghi- | silver beet; endive | of the leaves and sometimes on the |
| sweet corn; Uromyces | | pods as well; dusty reddish-brown |
| appendiculatus- | | spores released from pustules (may |
| beans; Puccinia allii- | | be black in cold weather). |
| spring onions). | | |
| Black root rot (Different | Lettuce; beans; cucurbits | Blackening of roots; stunted plants; |
| species on different | | plants may die. |
| vegetable crops) | | |

Other fungal diseases of vegetables, and the causative organism, include: target spot—Alternaria solani (tomatoes); Phanomyces root rot—Aphanomyces euteiches pv. phaseoli (beans); Aschochyta collar rot (peas); gummy stem blight—Didymella bryoniae (cucurbits); Alternaria leaf spot—Alternaria cucumerina and A. alternata (cucurbits); black leg—Leptosphaeria maculans (brassicas); ring spot—Mycosphaerella brassicicola (brassicas); late blight—Septoria apiicola (celery); Cercospora leaf spot—Cercospora beticola (beets); leaf blight—Septoria petroelini (parsley); Septoria spot—Septoria lactucae (lettuce); leaf blight of spring onions—Stemphylium vesicarium; and leaf blight of carrots—Alternaria dauci. Control and management of fungal diseases can be benefited with a good understanding of the fungi, the periods during which the crops are susceptible, and the influence of environmental conditions.

Fungi also infect a wide range of monocot plants, including grasses such as agriculturally important grass species, and other commercially relevant crop plants. Thus, the herein provided biologically-based pesticides can be used to reduce or minimize the impact of fungal infections in monocotyledonous plants, such as rice, wheat, corn (maize), sorghum, and other agriculturally important monocot crops (such as palms, bananas and plantains, ginger and relatives, turmeric and cardamom, asparagus, pineapple, sedges and rushes, and alliums (e.g., leeks, onion, shallots, and garlic)).

By way of example, Fusarium oxysporum causes wilting on many monocotyledonous plants. The corresponding symptoms include stunting, yellowing of the leaves, defoliation, downward bending of the leaves (leaf epinasty), loss of green coloration of plant veins (vein clearing).

Fungal diseases of wheat include: wheat leaf rust or brown rust, which caused by Puccinia triticina. Wheat stem rust or black rust is caused by Puccinia graminis f. sp. tritici. Stipe rust (also known as yellow rust) is caused by Puccinia striiformis f. sp. Tritici. Powdery mildew is caused by Blumeria graminis f. sp. Tritici. Karnal bunt is caused by Tilletia indica. Flag smut is caused by Urocystis agropyri. Common bunt of wheat is caused by Tilletia caries. Pyrenophora tritici repentis causes tan spot in wheat. Septoria is a disease complex caused by three different pathogens: P. avenaria triticae, Mycosphaerella graminicola, and Phaeosphaeria nodorum. Common root rot, crown root rot, and black point are caused by Bipolaris sorokiniana.

Fungal disease organisms and resultant diseases of corn (maize) include: Cercospora zeae-maydis, which causes grey leaf spot.; Puccinia sorghi, which causes common rust; Colletotirchum graminicola, which causes anthracnose. Kabatiella zeae, which causes corn eyespot disease, and Bipolaris maydis, which causes Southern corn leaf blight.

One of the most destructive diseases of rice is rice blast, caused by Magnaporthe grisea (Pyricularia oryzae). Also in rice, sheath blight is caused by the fungus Thanatephorus cucumeris; sheath rot is caused by Acrocylindrium oryzae. Leaf scald is caused by Metasphaeria albescens; false smut is caused by Claviceps virens; and kernel smut is caused by Tilletia barclayana.

The MEL-containing compositions described herein (including formulations and compositions that are prepared for application to a plant or field, for instance in an agricultural setting) can be examined and characterized using any art-recognized systems for detecting and/or measuring characteristics that may influence the function or behavior of the formulation. These characteristics may include, for instance, solubility, viscosity, pH, density, stability (including short term, long term, and at various temperatures), bloom, dispersibility, re-dispersibility, sprayability, drift, coverage, efficacy (including in the field), and so forth. Standard methods for making such measurements can be found in the Collaborative International Pesticides Analytical Council (CIPAC) Handbooks, which can be accessed online (cipac.org/index.php/methods-publications/handbooks).

### Uses of Biopesticide Compositions

Methods of preparing compositions for application in agricultural settings is well known, including for instance preparation and application of compositions that include a biocide such as the herein-described MEL-containing biopesticide compositions. The application of the provided MEL-containing biological pesticides is generally conventional, and appropriate methods will be known to those of skill in the art. See, for instance: WO 2021/055311, US 2005/0096393, U.S. Pat. No. 11,284,620, US 2012/0027741, US 2018/0281819, WO 2019/177174. Additional representative discussion is provided herein.

For instance, there are provided herein compositions that are concentrates—that is, formulations that contain an active ingredient (such as a MEL-containing biological pesticide) at level higher than the as-applied level of that ingredient—which concentrates are intended to be diluted before application or use. Concentrates are recognized as beneficial, for instance because they can be more efficiently stored (since they take up less volume than a diluted formulation), they are in various instances more stable for long-term storage or shipment, and so forth. However, it is important that concentrate formulations are diluted before use in order to avoid waste, to avoid toxicity that may result from using active ingredient(s) or other components at a higher level than recommended, to avoid phytotoxicity effects arising from mis-balanced formulation components, and to avoid environmental contamination and/or user health impacts. The art recognizes methods for diluting concentrate formulations; the following discussion is provided for guidance only, and is not intended to be limiting.

In particular embodiments, compositions of the present disclosure are used to protect a plant and/or a plant part from an infectious agent, such as a fungal infection.

The success of use of a MEL-containing bio-fungicide (biopesticide) composition as described herein can be examined using any of myriad known measures of improved plant survival, growth and/or production, as well as reduction of visible or measurable disease presence or progression. For instance, representative indirect measurements of reduced disease presence include measuring plant yield, plant biomass, shoot biomass, shoot length, dry shoot weight, fresh shoot weight, seedling shoot length, dry seedling weight, fresh seedling weight, leaf surface area, number of stomata per leaf, root biomass, root length, root surface area, germination rate, emergence rate, chlorophyll content, vigor, seed yield, dry weight of mature seeds, fresh weight of mature seeds, number of mature seeds per plant, number of pods per plant, length of pods per plant, plant height, or a combination of any two or more thereof.

### Representative Methods of Field Application

Once the appropriate concentration of MEL-based biopesticide formulation (an RTU or “as-applied” composition) has been prepared, the formulation may be deposited on soil in which plants or crops are being planted, grown, harvested or any combination of the preceding or directly on the plants during any stage of growth. Methods of distributing or applying the MEL-based biopesticide formulations may include broadcast spraying or spreading or directed application. Broadcast spreading typically is used when a product needs to be distributed over a larger area such as across a field which enables the product to spread across the field. Broadcast spreading may take various forms such as via hand-held sprayer, tractor, aircraft, or other means. In contrast, directed application is normally used when there is a desire to apply the product to a specific area of the field or crops. Directed applications may be applied via tractor or other depositing device.

By way of example, the MEL-based biopesticide formulation may be deposited in a tank or other container. The tank may then be sealed and optionally pressurized at which point the tank may be connected to any desired distribution device (e.g. sprayer, tractor, or aircraft) and administered to the soil or crops as desired. Alternatively, the product could be administered sub-soil via injection prior to or at the time a field is seeded. An additional method may involve mixing the product with irrigation water wherein the product is distributed at the time of irrigation.

In one embodiment or in combination with any of the mentioned embodiments, the MEL-based biopesticide formulation is applied to edible plant parts, such as leaves, stems, roots, corms, bulbs, rhizomes, fruits, and/or vegetables. Such application can occur at any time during the plant growth cycle, depending on the active ingredient(s) being applied and the field application conditions. In particular embodiments, the MEL-based biopesticide formulation is applied prior to or at the bud stage, prior to or at the flowering stage or once the fruit as started to or has developed or anytime during any of these time periods. The MEL-based biopesticide formulation may be applied, for example, by spraying.

Aspects of the current disclosure are now described with additional details and options. Headings do not limit the interpretation of the disclosure and are provided for organizational purposes only.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Exemplary Embodiments

1. A composition including mannosylerythritol lipid (MEL), produced by fungal fermentation in a growth medium including: at least one carbon source that includes an oil, a yeast extract, sodium nitrite, potassium dihydrogen phosphate, and magnesium sulfate heptahydrate.

2. The composition of embodiment 1, wherein the carbon source includes lignocellulose and its hydrolysate, vegetable oil, and glucose.

3. The composition of embodiment 1 or 2, wherein the growth medium further includes calcium chloride dihydrate, ferrous sulfate heptahydrate, and manganese sulfate.

4. The composition of any of embodiments 1-3, wherein the growth medium includes: 7% to 20% by weight of the carbon source; 0.5% to 2% by weight of sodium nitrate; 0.1% to 0.3% by weight of yeast extract; 0.3% to 0.7% by weight of potassium dihydrogen phosphate; 0.03% to 0.07% by weight of magnesium sulfate heptahydrate; 0.005% to 0.015% by weight of calcium chloride dihydrate; 0.005% to 0.015% by weight of ferrous sulfate heptahydrate; and 0.0005% to 0.0015% by weight of manganese sulfate.

5. The composition of any of embodiments 1-4, wherein the growth medium includes: 15% by weight of the carbon source; 1% by weight of sodium nitrate; 0.2% by weight of yeast extract; 0.5% by weight of potassium dihydrogen phosphate; 0.05% by weight of magnesium sulfate heptahydrate; 0.01% by weight of calcium chloride dihydrate; 0.01% by weight of ferrous sulfate heptahydrate; and 0.001% by weight of manganese sulfate.

6. The composition of any of embodiments 1-4, wherein the growth medium includes: 10% by weight of the carbon source; 0.5% by weight of sodium nitrate; 0.3% by weight of yeast extract; 0.25% by weight of potassium dihydrogen phosphate; 0.05% by weight of magnesium sulfate heptahydrate; 0.005% by weight of calcium chloride dihydrate; 0.005% by weight of ferrous sulfate heptahydrate; and 0.002% by weight of manganese sulfate.

7. The composition of any of embodiments 1-4, wherein the growth medium includes: 9% by weight of the carbon source; 0.5% by weight of sodium nitrate; 0.3% by weight of yeast extract; 0.25% by weight of potassium dihydrogen phosphate; 0.05% by weight of magnesium sulfate heptahydrate; 0.005% by weight of calcium chloride dihydrate; 0.005% by weight of ferrous sulfate heptahydrate; and 0.002% by weight of manganese sulfate.

8. The composition of any of embodiments 1-4, wherein the growth medium includes: 12% by weight of the carbon source; 0.4% by weight of sodium nitrate; 0.3% by weight of yeast extract; 0.25% by weight of potassium dihydrogen phosphate; 0.05% by weight of magnesium sulfate heptahydrate; 0.005% by weight of calcium chloride dihydrate; 0.005% by weight of ferrous sulfate heptahydrate; and 0.002% by weight of manganese sulfate.

9. The composition of any of embodiments 1-8, wherein the fungal microbe used to produce the MEL includes at least one selected from the group consisting of genus Pseudozyma (Moesziomyces).

10. The composition of embodiment 1, produced by fermentation of a Pseudozyma fungus in a growth medium including: 15% by weight of carbon source including lignocellulose and its hydrolysate, vegetable oil, and glucose, wherein the mass ratio of the lignocellulose and hydrolysate:vegetable oil:glucose is 1:6:3; 1% by weight of sodium nitrate; 0.2% by weight of yeast extract; 0.5% by weight of potassium dihydrogen phosphate; 0.05% by weight of magnesium sulfate heptahydrate; 0.01% by weight of calcium chloride dihydrate; 0.01% by weight of ferrous sulfate heptahydrate; and 0.001% by weight of manganese sulfate.

11. The composition of embodiment 1, produced by fermentation of a Pseudozyma fungus in a growth medium including: 10% by weight of carbon source including lignocellulose and its hydrolysate, vegetable oil, and glucose, wherein the mass ratio of the lignocellulose and hydrolysate:vegetable oil:glucose is 1:7:2; 0.5% by weight of sodium nitrate; 0.3% by weight of yeast extract; 0.25% by weight of potassium dihydrogen phosphate; 0.05% by weight of magnesium sulfate heptahydrate; 0.005% by weight of calcium chloride dihydrate; 0.005% by weight of ferrous sulfate heptahydrate; and 0.002% by weight of manganese sulfate.

12. The composition of embodiment 1, produced by fermentation of a Pseudozyma fungus in a growth medium including: 9% by weight of the carbon source including lignocellulose and its hydrolysate, vegetable oil, and glucose, wherein the mass ratio of the lignocellulose and hydrolysate:vegetable oil:glucose is 0.3:0.8:1.7; 0.5% by weight of sodium nitrate; 0.3% by weight of yeast extract; 0.25% by weight of potassium dihydrogen phosphate; 0.05% by weight of magnesium sulfate heptahydrate; 0.005% by weight of calcium chloride dihydrate; 0.005% by weight of ferrous sulfate heptahydrate; and 0.002% by weight of manganese sulfate.

13. The composition of embodiment 1, produced by fermentation of a Pseudozyma fungus in a growth medium including: 12% by weight of the carbon source including lignocellulose and its hydrolysate, vegetable oil, and glucose, wherein the mass ratio of the lignocellulose and hydrolysate:vegetable oil:glucose is 0.3:9.2:0.4; 0.4% by weight of sodium nitrate; 0.3% by weight of yeast extract; 0.25% by weight of potassium dihydrogen phosphate; 0.05% by weight of magnesium sulfate heptahydrate; 0.005% by weight of calcium chloride dihydrate; 0.005% by weight of ferrous sulfate heptahydrate; and 0.002% by weight of manganese sulfate.

14. A biological pesticide including the composition of any one of embodiments 1-13.

15. A process for preparing a mannosylerythritol lipid (MEL) composition made by microbial fermentation, including: inoculating a first growth medium including at least one carbon source, sodium nitrite, yeast extract, potassium dihydrogen phosphate, and magnesium sulfate heptahydrate with a seed liquid; wherein the seed liquid is made by inoculating a second growth medium including yeast extract, peptone, glucose, maltose and sodium chloride and water with a microbe and culturing said inoculated second growth medium; culturing said inoculated first growth medium to produce a fermentation broth; temperature sterilizing the fermentation broth to obtain a sterilized fermentation broth; filtering the sterilized fermentation broth, to produce a filtrate; adjusting the pH of the filtrate to an acidic pH to obtain a microbe separation liquid wherein the microbe separation liquid is miscible in an least one organic solvent; and extracting the microbe separation liquid with the at least one organic solvent, to produce the MEL composition.

16. The process of embodiment 15, wherein the first growth medium further includes calcium chloride dihydrate, ferrous sulfate heptahydrate, and manganese sulfate.

17. The process of embodiment 15 or 16, wherein the first growth medium includes: 7% to 20% by weight of the carbon source; 0.5% to 2% by weight of sodium nitrate; 0.1% to 0.3% by weight of yeast extract; 0.3% to 0.7% by weight of potassium dihydrogen phosphate; 0.03% to 0.07% by weight of magnesium sulfate heptahydrate; 0.005% to 0.015% by weight of calcium chloride dihydrate; 0.005% to 0.015% by weight of ferrous sulfate heptahydrate; and 0.0005% to 0.0015% by weight of manganese sulfate.

18. The process of embodiment 17, wherein the first growth medium includes: 15% by weight of the carbon source; 1% by weight of sodium nitrate; 0.2% by weight of yeast extract; 0.5% by weight of potassium dihydrogen phosphate; 0.05% by weight of magnesium sulfate heptahydrate; 0.01% by weight of calcium chloride dihydrate; 0.01% by weight of ferrous sulfate heptahydrate; and 0.001% by weight of manganese sulfate.

19. The process of embodiment 17, wherein the first growth medium includes: 10% by weight of the carbon source; 0.5% by weight of sodium nitrate; 0.3% by weight of yeast extract; 0.25% by weight of potassium dihydrogen phosphate; 0.05% by weight of magnesium sulfate heptahydrate; 0.005% by weight of calcium chloride dihydrate; 0.005% by weight of ferrous sulfate heptahydrate; and 0.002% by weight of manganese sulfate.

20. The process of embodiment 17, wherein the first growth medium includes: 9% by weight of the carbon source; 0.5% by weight of sodium nitrate; 0.3% by weight of yeast extract; 0.25% by weight of potassium dihydrogen phosphate; 0.05% by weight of magnesium sulfate heptahydrate; 0.005% by weight of calcium chloride dihydrate; 0.005% by weight of ferrous sulfate heptahydrate; and 0.002% by weight of manganese sulfate.

21. The process of embodiment 17, wherein the first growth medium includes: 12% by weight of the carbon source; 0.4% by weight of sodium nitrate; 0.3% by weight of yeast extract; 0.25% by weight of potassium dihydrogen phosphate; 0.05% by weight of magnesium sulfate heptahydrate; 0.005% by weight of calcium chloride dihydrate; 0.005% by weight of ferrous sulfate heptahydrate; and 0.002% by weight of manganese sulfate.

22. The process of any of embodiments 15-22, wherein the second growth medium includes: 0.2% to 0.4% by weight of yeast extract; 0.4% to 0.8% by weight of peptone; 0.2% to 0.6% by weight of glucose; 0.05% to 0.15% by weight of maltose; and 0.3% to 0.7% by weight of sodium chloride.

23. The process of embodiment 22, wherein the second growth medium includes: 0.3% by weight of yeast extract; 0.6% by weight of peptone; 0.4% by weight of glucose; 0.1% by weight of maltose; and 0.5% by weight of sodium chloride.

24. The process of embodiment 22 or embodiment 23, wherein the remainder of the growth medium is water.

25. The process of any of embodiments 15-24, further including maintaining the inoculated first growth medium at a temperature of from 22° C. to 32° C.

26. The process of embodiment 25, wherein the inoculated first growth medium is maintained at a temperature of from 25° C. to 28° C.

27. The process of any of embodiments 15-26, further including stirring the inoculated first growth medium.

28. The process of any of embodiments 15-27, wherein the inoculated first growth medium is cultured for from 5 days to 15 days.

29. The process of embodiment 28, wherein the inoculated first growth medium is cultured for 10 days.

30. The process of any of embodiments 15-29, further including maintaining the second inoculated growth medium at a temperature of from 25° C. to 35° C.

31. The process of embodiment 30, wherein the second inoculated growth medium is maintained at a temperature of 30° C.

32. The process of any of embodiments 15-31, wherein the inoculated second growth medium is cultured for from 1 day to 3 days.

33. The process of any of embodiments 15-32, wherein the temperature sterilization is carried out at: a temperature of from 60° C. to 140° C.; or a temperature of from 80° C. to 120° C.

34. The process of any of embodiments 15-33, wherein the temperature for the temperature sterilization is maintained for a period for from 1 hour to 3 hours.

35. The process of embodiment 34, wherein the temperature for the temperature sterilization is maintained for 2 hours.

36. The process of according to any of embodiments 15-35, wherein the filtrate is maintained at a pH of from 2 to 5.

37. The process of embodiment 36, wherein the filtrate is maintained at a pH of less than 3.

38. The process of any of embodiments 15-37, wherein the filtrate is maintained at a temperature of from 4° C. to 10° C.

39. The process of embodiment 38, wherein the temperature of from 4° C. to 10° C. is maintained from 12 hours to 36 hours.

40. The process of embodiment 39, wherein the temperature of from 4° C. to 10° C. is maintained for 24 hours.

41. The process of any of embodiments 15-40, wherein the at least one organic solvent includes ethyl acetate or chloroform.

42. The process of any of embodiments 15-41, further including isolating the organic phase from the extraction of the microbe separation liquid.

43. The process of embodiment 42, further including removing the organic solvent from the organic phase to provide a concentrated microbe separation liquid.

44. An MEL composition made by the process of any of embodiments 15-43.

45. A biological pesticide including the MEL composition of embodiment 44.

46. A method of preventing or treating a crop disease caused by a crop pathogen, including contacting the crop infected by the crop pathogen with a therapeutically effective amount of the biopesticide of embodiment 14 or embodiment 45.

47. The method of embodiment 46, wherein the crop disease includes rice blast, Sorghum hard smut, rice smut, corn leaf blight, and other fungal diseases.

48. The method of embodiment 47, wherein the crop disease is rice disease.

49. The method of embodiment 48, wherein the rice disease is rice blast, or rice false smut disease.

50. The method of embodiment 46, wherein the crop is selected from the group consisting of: rice, wheat, corn, sorghum, and other commercially relevant crop plants.

### Example 1: Pseudozyma Biopesticide Fermented from Lignocellulose

This example describes a process for making a fermented biopesticide composition using black powder fungus Pseudozyma microorganism. The resultant composition is shown to be effective against a representative crop organism.

Seed Preparation: Colonies were picked from a solid culture plate of Pseudozyma, and inoculated into the following medium: yeast powder 0.3%, peptone 0.6%, glucose 0.4%, maltose 0.1%, sodium chloride 0.5%, with the remainder being made of water. The inoculated liquid culture was then transferred to a constant temperature at 30° C., cultured for 1-3 days, to obtain the seed liquid.

Synthesis: Inoculum size of 8% volume of this Pseudozyma seed liquid was transferred to the prepared culture medium, with raw material comprising lignocellulose and its hydrolysate, vegetable oil, glucose as main carbon source. The culture was the cultivated under at 1.0 vvm aeration, with the temperature controlled at 25-28° C., and stirred at 200 rpm. The medium contained: carbon source 15% (lignocellulose and its hydrolysate:vegetable oil:glucose=1:6:3, mass ratio), sodium nitrate 1%, yeast powder 0.2%, potassium dihydrogen phosphate 0.5%, magnesium sulfate heptahydrate 0.05%, calcium chloride dihydrate 0.01%, ferrous sulfate heptahydrate 0.01%, manganese sulfate 0.001%. After culturing for 10 days, the fermentation broth of the microbe was obtained.

Separation: The fermentation broth was subjected to high temperature sterilization at 80-120° C. for 2 hours. The sterilized fermentation broth was filtered to remove microbial cells; the filtration method can employ membranes or filter bags, for instance. The resultant liquid after microbial cells were removed was then subject to high-speed centrifugation to remove residual starting material. The liquid obtained after centrifugation collected, adjusted to pH of 3 or lower, and held at a temperature of 4-10° C. refrigerator at 24 hours.

Extraction and concentration: The Pseudozyma separation liquid was extracted with ethyl acetate, and the organic phase was collected. The liquid obtained by this separation was fully mixed with solvents of different volumes, and then allowed to stand for separation. It was then concentrated 2-5 times in vacuo at 40-60° C.; the solvent can be collected for recycling. The process can also be extracted through organic or ceramic membranes, followed by vacuum concentration at 40-60° C.

Application test: The biopesticide obtained in the above process was used for crop disease control test. This was illustrated by preparing solid medium plates, diluting the biological pesticide to a certain multiple (200, 2000, 20000 fold), spreading it evenly on the surface of the solid medium, and then the crop pathogen (Magnaporthe grisea, rice blast) was spotted in the center of the treated plate. The plate was placed in an incubator to observe the inhibitory function of the biopesticide preparation.

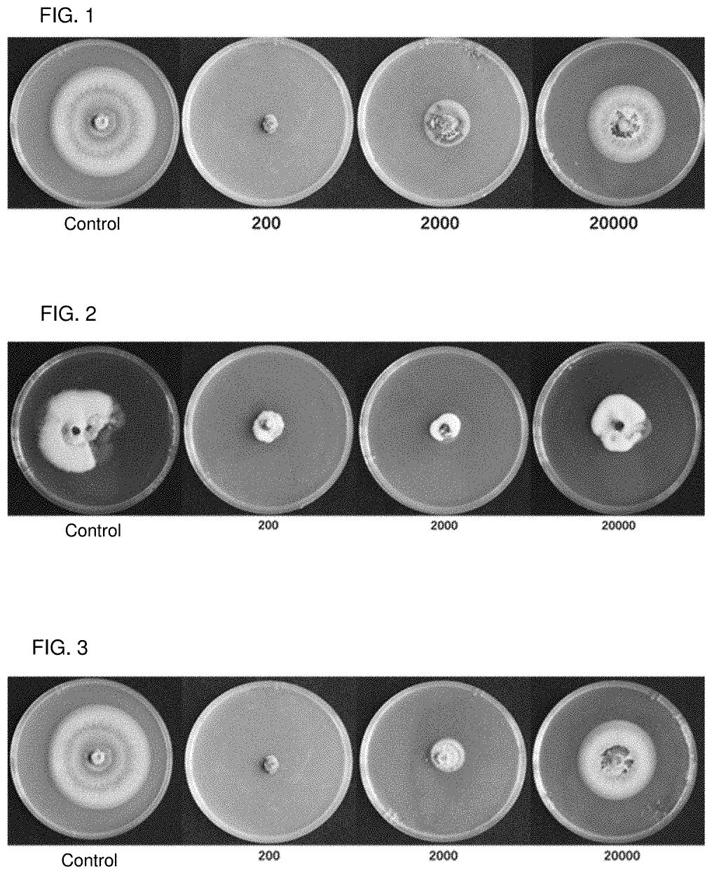

From the results shown in Table 1 and FIG. 1, it can be seen that after the biopesticide has been diluted by a high multiple, it still has a high inhibition efficiency. Even with 2000 fold dilution, it still maintains an effective inhibition rate of more than 70%.

TABLE 1
Inhibitory effects
of biological pesticides
on Magnaporthe grisea

| fold dilution | Inhibition rate % |
|---|---|
| 200 times | 93.07 |
| 2000 times | 74.34 |
| 20000 times | 56.54 |

### Example 2: Ustilaginales Biopesticide Fermented from Lignocellulose

This example describes a process for making a fermented biopesticide composition using black powder fungus Ustilaginales microorganism. The resultant composition is shown to be effective against a representative crop organism.

Seed preparation: Colonies were picked from a solid culture plate of Ustilaginales and inoculated it into the following medium: yeast powder 0.3%, peptone 0.6%, glucose 0.4%, maltose 0.1%, sodium chloride 0.5%, with the remainder being made of water. The inoculated liquid culture was then transferred to a constant temperature at 30° C. and cultured for 1-3 days to obtain the seed liquid.

Synthesis: Inoculum size of 10% volume of this Ustilaginales seed liquid was transferred to the prepared culture medium, with raw material including lignocellulose and its hydrolysate, vegetable oil, glucose etc. as the main carbon source. The culture was the cultivated under 1.0 vvm aeration, with the temperature controlled at 25-28° C., and stirred at 200 rpm. The medium contained: carbon source 10% (lignocellulose and its hydrolysate:vegetable oil:glucose=1:7:2, mass ratio), sodium nitrate 0.5%, yeast powder 0.3%, potassium dihydrogen phosphate 0.25%, Magnesium sulfate heptahydrate 0.05%, calcium chloride dihydrate 0.005%, ferrous sulfate heptahydrate 0.005%, manganese sulfate 0.002%. After culturing for 14 days, the fermentation broth of the microbe was obtained.

Separation: The fermentation broth was subjected to high temperature sterilization at 80-120° C. for 2 hours. The sterilized fermentation broth was filtered to remove microbial cells; the filtration method can employ membranes or filter bags, for instance. The resultant liquid after microbial cells were removed was then subject to high-speed centrifugation to remove residual starting material. The liquid obtained after centrifugation collected, adjusted to pH of 3 or lower, and held at a temperature of 4-10° C. refrigerator at 24 hours.

Extraction and concentration: The Ustilaginales separation liquid was extracted with ethyl acetate, and the organic phase was collected. The liquid obtained by this separation was fully mixed with different volumes of solvents, and then allowed to stand for separation. It was then concentrated 2-5 times in vacuo at 40-60° C.; the solvent may be collected for recycling. Using a combination of organic membrane and inorganic ceramic membrane, with a membrane molecular weight cut-off of 20 KD-100 KD, the obtained filtrate was vacuum concentrated 5-10 times at 40-60° C., and the solvent collected for recycling. The process can also be extracted through organic or ceramic membranes, followed by vacuum concentration at 40-60° C.

Application test: The biopesticide obtained in the above process was used for crop disease control test. This was illustrated by preparing solid medium plates, diluting the biological pesticide to a certain multiple (200, 2000, 20000 times), spreading it evenly on the surface of the solid medium, and then the crop pathogen (Ustilaginoidea virens (Cke), Tak, green smut) was spotted in the center of the treated plate. The plate was placed in an incubator to observe the inhibitory function of the biopesticide preparation.

From the results shown in Table 2 and FIG. 2, it can be seen that after the biopesticide has been diluted by a high multiple, it still has a high inhibition efficiency. Even with 2000 fold dilution, it still maintains an effective inhibition rate of more than 75%.

TABLE 2
Inhibitory effect
of biological pesticides
on Ustilaginoidea virens

| fold dilution | Inhibition rate % |
|---|---|
| 200 times | 91.62 |
| 2000 times | 79.92 |
| 20000 times | 42.53 |

### Example 3: Moesziomyces Biopesticide Fermented from Lignocellulose

This example describes a process for making a fermented biopesticide composition using black powder fungus Moesziomyces microorganism. The resultant composition is shown to be effective against a representative crop organism

Seed preparation: Colonies were picked from a solid culture plate of Moesziomyces fungus and inoculated into the following medium: yeast powder 0.3%, peptone 0.6%, glucose 0.4%, maltose 0.1%, sodium chloride 0.5 with the remainder being made of water. The inoculated liquid culture was then transferred to a constant temperature at 30° C., and cultured for 1-3 days to obtain the seed liquid.

Synthesis: Inoculum size of 5% volume of this Moesziomyces seed liquid was transferred to the prepared culture medium with raw material comprising lignocellulose hydrolysate, vegetable oil, and glucose as the main carbon source. The culture was the cultivated under 1.0 vvm aeration, with the temperature controlled at 25-28° C., and stirred at 200 rpm. The medium contained: carbon source 9% (lignocellulose and its hydrolysate:vegetable oil:glucose=0.3:8:1.7, mass ratio), sodium nitrate 0.5%, yeast powder 0.3%, potassium dihydrogen phosphate 0.25%, Magnesium sulfate heptahydrate 0.05%, calcium chloride dihydrate 0.005%, ferrous sulfate heptahydrate 0.005%, manganese sulfate 0.002%. After culturing for 14 days, the fermentation broth of the microbe was obtained.

Separation: The fermentation broth was subjected to high temperature sterilization at 80-120° C. for 2 hours. The sterilized fermentation broth was filtered to remove microbial cells; the filtration method can employ membranes or filter bags, for instance. The resultant liquid after microbial cells were removed was then subject to high-speed centrifugation to remove residual starting material. The liquid obtained after centrifugation collected, adjusted to pH of 3 or lower, and held at a temperature of 4-10° C. refrigerator at 24 hours.

Extraction and concentration: Moesziomyces, etc. are used to separate the liquids with ethyl acetate, and then the organic phase is collected. The liquids obtained by this separation were fully mixed with solvents of different volumes, and then allowed to stand for separation. The Pseudomonas separation liquid was extracted using a combination of organic membrane and inorganic ceramic membrane. The molecular weight cut-off of the membrane was 20 KD-100 KD. The obtained filtrate was then vacuum concentrated 5-10 times at 40-60° C., and the solvent collected for recycling. The process can also be extracted through organic or ceramic membranes, followed by vacuum concentration at 40-60° C.

Application test: The biopesticide obtained in the above process was used for crop disease control test. This was illustrated by preparing solid medium plates, diluting the biological pesticide to a certain multiple (200, 2000, 20000 times), spreading it evenly on the surface of the solid medium, and then the crop pathogen (Magnaporthe oryzae, rice fever fungus) was spotted in the center of the treated plate. The plate was placed in an incubator to observe the inhibitory function of the biopesticide preparation.

From the results shown in Table 3 and FIG. 3, it can be seen that after the biopesticide has been diluted by a high multiple, it still has a high inhibition efficiency. Even with 2000 fold dilution, it still maintains an effective inhibition rate of more than 80%.

TABLE 3
Inhibitory effects
of biological pesticides
on Magnaporthe oryzae

| fold dilution | Inhibition rate % |
|---|---|
| 200 times | 93.02 |
| 2000 times | 82.35 |
| 20000 times | 47.39 |

### Example 4: A Multiple-Microbe Biopesticide Composition Fermented from Starch

This example describes a process for making a fermented biopesticide composition using more than one starting microorganism. The resultant composition is shown to be effective against a representative crop organism, even at very high dilution.

Seed preparation: Colonies were picked from solid culture plates of Pseudozyma, Ustilaginales, Moesziomyces, etc. and inoculated into the following medium: yeast powder 0.3%, peptone 0.6%, glucose 0.4%, maltose 0.1%, sodium chloride 0.5%, with the remainder being made of water. The inoculated liquid cultures were then transferred to a constant temperature at 30° C. and cultured for 1-3 days, to obtain the seed liquid of each strain.

Synthesis: Inoculum size of 5% volume of these three seed liquids (e.g., Pseudozyma, Ustilaginales, Moesziomyces) were respectively transferred to prepared culture medium, with raw material comprising starch and its hydrolysate, vegetable oil, glucose as the main carbon source. These were then cultured at 1.0 vvm aeration, with the temperature controlled at 26-30° C., and stirred at 300 rpm. The medium was: carbon source 12% (starch hydrolysate:vegetable oil:glucose=0.3:9.2:0.4, mass ratio), sodium nitrate 0.4%, yeast powder 0.3%, potassium dihydrogen phosphate 0.25%, magnesium sulfate heptahydrate 0.05%, calcium chloride dihydrate 0.005%, ferrous sulfate heptahydrate 0.005%, manganese sulfate 0.002%. After culturing for 14 days, the fermentation broth of each microbe was obtained.

Separation: Each fermentation broth obtained in this fermentation process was subjected to high temperature sterilization at 80-120° C. for 2 hours. The sterilized fermentation broth was filtered to remove microbial cells; the filtration method can employ membranes or filter bags, for instance. The resultant liquid after cells were removed was then subject to high-speed centrifugation to remove residual starting material. The liquid obtained after centrifugation was collected, adjusted to pH of 3 or lower, and held at a temperature of 4-10° C. refrigerator at 24 hours.

Extraction and concentration: Separate fermentation liquids such as those produced by fermentation of Pseudozyma, Ustilaginales, Moesziomyces, etc. were extracted with ethyl acetate, and then the organic phase was collected. The liquids obtained by this separation were fully mixed with solvents of different volumes, and then allowed to stand for separation. The separation liquid was extracted by a combination of organic membrane and inorganic ceramic membrane. The molecular weight cut-off of the membrane is 20 KD-100 KD. The obtained filtrate was then vacuum concentrated 5-10 times at 40-60° C.; the solvent can be collected for recycling. The process can also be extracted through organic or ceramic membranes, followed by vacuum concentration at 40-60° C.

Mixing: The three fermentation broths separated in this manner were mixed according to the desired proportion. In this example, Pseudozyma, Ustilaginales, and Moesziomyces preparations were mixed in a ratio of 1:1:1 to obtain biological pesticide products.

Application test: The biopesticide obtained in the above process was used for crop disease control test. This was illustrated by preparing solid medium, diluting the biological pesticide to a certain multiple (illustrated with a 2000-fold dilution), spreading it evenly on the surface of the solid medium plate, and then the crop pathogen (Bipolaris maydis, Xiaoban disease) was spotted in the center of the treated plate. The plate was placed in an incubator to observe the inhibitory function of the biopesticide preparation.

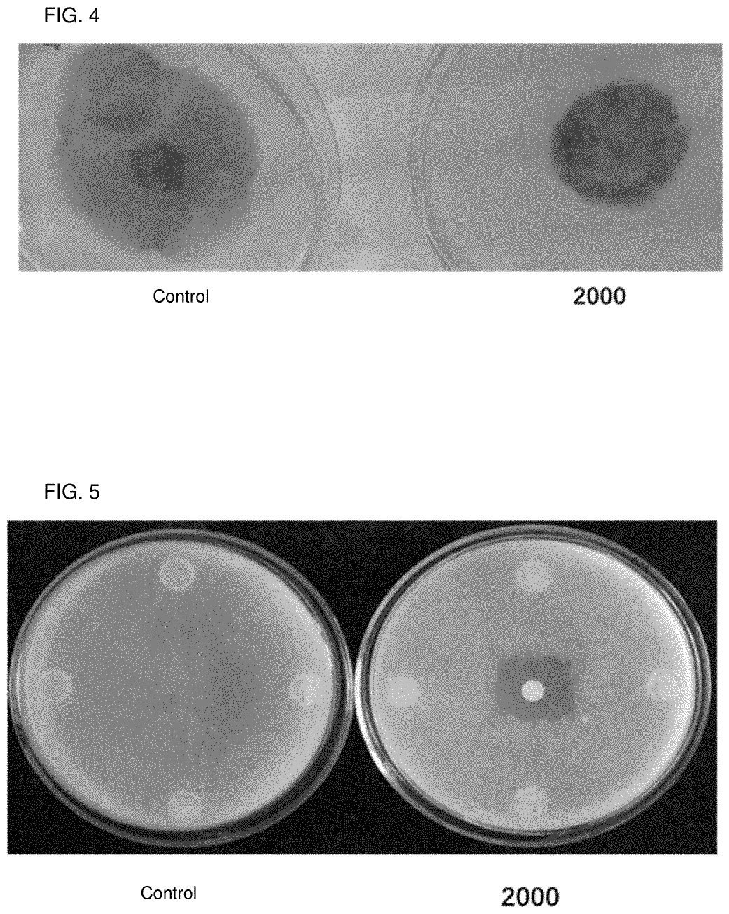

From the results in FIG. 4, it can be seen that after the biopesticide was diluted by a high fold (2000×), it still has a high inhibition efficiency. Even with 2000 fold dilution, it still maintains an effective inhibition rate of more than 60%.

### Example 5: A Multiple-Microbe Biopesticide Composition Fermented from Starch

This example describes a process for making a fermented biopesticide composition using more than one starting microorganism. The resultant composition is shown to be effective against a representative crop organism, even at very high dilution.

Seed preparation: Colonies were picked from solid culture plates of Pseudozyma, Ustilaginales, Moesziomyces (as exemplified here), and inoculated into the following medium: yeast powder 0.3%, peptone 0.6%, glucose 0.4%, maltose 0.1%, sodium chloride 0.5%, with the remainder being made of water. The inoculated liquid cultures were then transferred to a constant temperature at 30° C. and cultured for 1-3 days, to obtain the seed liquid of each strain.

Synthesis: Inoculum size of 5% volume of these three seed liquids (e.g., Pseudozyma, Ustilaginales, Moesziomyces) were respectively transferred to prepared culture medium, with raw material comprising starch and its hydrolysate, vegetable oil, glucose as the main carbon source. These were then cultured at 1.0 vvm aeration, with the temperature controlled at 26-30° C., and stirred at 300 rpm. The culture medium contained: carbon source 10% (starch hydrolysate:vegetable oil:glucose=0.3:9.2:0.4, mass ratio), sodium nitrate 0.4%, yeast powder 0.3%, potassium dihydrogen phosphate 0.25%, magnesium sulfate heptahydrate 0.05%, calcium chloride dihydrate 0.005%, ferrous sulfate heptahydrate 0.005%, manganese sulfate 0.002%. After culturing for 14 days, the fermentation broth of each microbe was obtained.

Separation: Each fermentation broth obtained in this fermentation process was subjected to high temperature sterilization at 80-120° C. for 2 hours. The sterilized fermentation broth was filtered to remove microbial cells; the filtration method can employ membranes or filter bags, for instance. The resultant liquid after cells were removed was then subject to high-speed centrifugation to remove residual starting material. The liquid obtained after centrifugation was collected, adjusted to pH of 3 or lower, and held at a temperature of 4-10° C. refrigerator at 24 hours.

Extraction and concentration: Separate fermentation liquids such as those produced by fermentation of Pseudozyma, Ustilaginales, Moesziomyces, etc. were extracted with ethyl acetate, and then the organic phase was collected. The liquids obtained by this separation were fully mixed with solvents of different volumes, and then allowed to stand for separation. The separation liquid was extracted by a combination of organic membrane and inorganic ceramic membrane. The molecular weight cut-off of the membrane is 20 KD-100 KD. The obtained filtrate was then vacuum concentrated 5-10 times at 40-60° C.; the solvent can be collected for recycling. The process can also be extracted through organic or ceramic membranes, followed by vacuum concentration at 40-60° C.

Mixing: The three fermentation broths separated in this manner were mixed according to the desired proportion. Among them, three representative concentrates, such as those produced by Pseudozyma, Ustilaginales and Moesziomyces, were mixed at a ratio of 2:1:1 to obtain biological pesticide products.

Application test: The biopesticide obtained in the above process was used for crop disease control test. This was illustrated by preparing solid medium, diluting the biological pesticide to a certain multiple (illustrated with a 2000-fold dilution), spreading it evenly on the surface of the solid medium plate, and then the crop pathogen (Rizoctonia solani, sheath blight) was spotted in the center of the fixed plate. The plate was placed in an incubator to observe the inhibitory function of the biopesticide preparation.

From the results in FIG. 5, it can be seen that after the biopesticide was diluted by a high fold (2000×), it still showed a high inhibition efficiency. Even with 2000 fold dilution, it still maintained an effective inhibition rate of more than 90%.

### Example 6: Structural Characterization of Bio-Pesticide Preparation(s)

The structures of seven biologically active compounds produced as described in Examples 1-5 were identified by means of high performance liquid chromatography and mass spectrometry, gas chromatography and NMR, and it was concluded that they included but are not limited to yeast fermentation extracts such as Mannosylerythritol lipids (MELs), and fungal fermentation extracts.

### Example 7: Cost Comparison

This cost estimate includes the cost of raw materials, energy consumption, labor, equipment loss and other comprehensive costs. The cost of biopesticides produced by this method is compared with pesticides currently purchased in the market (such as tricyclazole fungicide (5-methyl-1,2,4-triazolo[3,4-b][1,3]benzothiazole; sold for instance as Blastin; see e.g., Peterson, L. G. (1990). Tricyclazole for Control of Pyricularia oryzae on Rice: the Relationship of the Mode of Action and Disease Occurrence and Development. In: Grayson, B. T., Green, M. B., Copping, L. G. (eds) Pest Management in Rice. Springer, Dordrecht. doi.org/10.1007/978-94 0775-1_8) or benzamide fungicides (such as benzoic acid amide and derivatives thereof; see e.g., Bartholomew et al., Synthesis and Chemistry of Agrochemicals III, ACS Symposium Series, 504, 1992, D01:10.1021/bk-1992-0504.ch040; and U.S. Pat. No. 4,808,628)).

The overall cost for herein-described biopesticide compositions is lower by 30%.

| Source of cost | Cost (RMB/ton) |
|---|---|
| This biological pesticide | 35000-40000 |
| Existing market | 40000-60000 |

The above examples illustrate that the present invention belongs to the application of synthetic biology and microbiology technology, the method for preparing biological pesticides is reasonable, and the synthetic biological pesticides have low cost, low carbon, environmental protection and safety.

The series of detailed descriptions listed above are only specific descriptions for the feasible embodiments of the present invention, and they are not intended to limit the protection scope of the present invention. Changes should all be included within the protection scope of the present invention.

### X. Closing Paragraphs

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in production and/or efficacy of a MEL-containing biopesticide preparation.

Use of the word(s), “exemplary” or “embodiment” or “desirably” in this document does not limit the definition or language with which the word(s) is used, and is intended to further illustrate in a non-limiting fashion meaning through use of an example or particular embodiments within the scope of the definition.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, other written text, and web site content throughout this specification (referenced materials herein). Each of the referenced materials are individually incorporated herein by reference in their entirety for their referenced teaching(s), as of the filing date of the first application in the priority chain in which the specific reference was included. For instance, with regard to chemical compounds, nucleic acid, and amino acids sequences referenced herein that are available in a public database, the information in the database entry is incorporated herein by reference as of the date of an application in the priority chain in which the database identifier for that compound or sequence was first included in the text.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art, such as the Oxford Dictionary of Biochemistry and Molecular Biology, 2^{nd} Edition (Ed. Anthony Smith, Oxford University Press, Oxford, 2006), and/or A Dictionary of Chemistry, 8^{th} Edition (Ed. J. Law & R. Rennie, Oxford University Press, 2020).

1. A composition comprising mannosylerythritol lipid (MEL), produced by fungal fermentation in a growth medium comprising:
at least one carbon source that comprises an oil,
a yeast extract,
sodium nitrite,
potassium dihydrogen phosphate, and
magnesium sulfate heptahydrate.

2. The composition of claim 1, wherein the carbon source comprises lignocellulose, a hydrolysate of lignocellulose, vegetable oil, and glucose.

3. (canceled)

4. The composition of claim 1, wherein the growth medium comprises:
7% to 20% by weight of the carbon source;
0.5% to 2% by weight of sodium nitrate;
0.1% to 0.3% by weight of yeast extract;
0.3% to 0.7% by weight of potassium dihydrogen phosphate;
0.03% to 0.07% by weight of magnesium sulfate heptahydrate;
0.005% to 0.015% by weight of calcium chloride dihydrate;
0.005% to 0.015% by weight of ferrous sulfate heptahydrate; and
0.0005% to 0.0015% by weight of manganese sulfate,
or
15% by weight of the carbon source;
1% by weight of sodium nitrate;
0.2% by weight of yeast extract;
0.5% by weight of potassium dihydrogen phosphate;
0.05% by weight of magnesium sulfate heptahydrate;
0.01% by weight of calcium chloride dihydrate;
0.01% by weight of ferrous sulfate heptahydrate; and
0.001% by weight of manganese sulfate;
or
10% by weight of the carbon source;
0.5% by weight of sodium nitrate;
0.3% by weight of yeast extract;
0.25% by weight of potassium dihydrogen phosphate;
0.05% by weight of magnesium sulfate heptahydrate;
0.005% by weight of calcium chloride dihydrate;
0.005% by weight of ferrous sulfate heptahydrate; and
0.002% by weight of manganese sulfate;
or
9% by weight of the carbon source;
0.5% by weight of sodium nitrate;
0.3% by weight of yeast extract;
0.25% by weight of potassium dihydrogen phosphate;
0.05% by weight of magnesium sulfate heptahydrate;
0.005% by weight of calcium chloride dihydrate;
0.005% by weight of ferrous sulfate heptahydrate; and
0.002% by weight of manganese sulfate;
or
12% by weight of the carbon source;
0.4% by weight of sodium nitrate;
0.3% by weight of yeast extract;
0.25% by weight of potassium dihydrogen phosphate;
0.05% by weight of magnesium sulfate heptahydrate;
0.005% by weight of calcium chloride dihydrate;
0.005% by weight of ferrous sulfate heptahydrate; and
0.002% by weight of manganese sulfate.

5-8. (canceled)

9. The composition of claim 1, wherein the fungal microbe used to produce the MEL is a member of the genus Moesziomyces (Pseudozyma) or the genus Ustilago.

10. The composition of claim 1, produced by fermentation of a Pseudozyma fungus in a growth medium comprising:
15% by weight of carbon source comprising lignocellulose and its hydrolysate, vegetable oil, and glucose, wherein the mass ratio of the lignocellulose and hydrolysate:vegetable oil:glucose is 1:6:3;
1% by weight of sodium nitrate;
0.2% by weight of yeast extract;
0.5% by weight of potassium dihydrogen phosphate;
0.05% by weight of magnesium sulfate heptahydrate;
0.01% by weight of calcium chloride dihydrate;
0.01% by weight of ferrous sulfate heptahydrate; and
0.001% by weight of manganese sulfate;
or
10% by weight of carbon source comprising lignocellulose and its hydrolysate, vegetable oil, and glucose, wherein the mass ratio of the lignocellulose and hydrolysate:vegetable oil:glucose is 1:7:2;
0.5% by weight of sodium nitrate;
0.3% by weight of yeast extract;
0.25% by weight of potassium dihydrogen phosphate;
0.05% by weight of magnesium sulfate heptahydrate;
0.005% by weight of calcium chloride dihydrate;
0.005% by weight of ferrous sulfate heptahydrate; and
0.002% by weight of manganese sulfate;
or
9% by weight of the carbon source comprising lignocellulose and its hydrolysate, vegetable oil, and glucose, wherein the mass ratio of the lignocellulose and hydrolysate:vegetable oil:glucose is 0.3:0.8:1.7;
0.5% by weight of sodium nitrate;
0.3% by weight of yeast extract;
0.25% by weight of potassium dihydrogen phosphate;
0.05% by weight of magnesium sulfate heptahydrate;
0.005% by weight of calcium chloride dihydrate;
0.005% by weight of ferrous sulfate heptahydrate; and
0.002% by weight of manganese sulfate;
or
12% by weight of the carbon source comprising lignocellulose and its hydrolysate, vegetable oil, and glucose, wherein the mass ratio of the lignocellulose and hydrolysate:vegetable oil:glucose is 0.3:9.2:0.4;
0.4% by weight of sodium nitrate;
0.3% by weight of yeast extract;
0.25% by weight of potassium dihydrogen phosphate;
0.05% by weight of magnesium sulfate heptahydrate;
0.005% by weight of calcium chloride dihydrate;
0.005% by weight of ferrous sulfate heptahydrate; and
0.002% by weight of manganese sulfate.

11-13. (canceled)

14. A biological pesticide comprising the composition of claim 1.

15. A process for preparing a mannosylerythritol lipid (MEL) composition made by microbial fermentation, comprising:
inoculating a first growth medium comprising at least one carbon source, sodium nitrite, yeast extract, potassium dihydrogen phosphate, and magnesium sulfate heptahydrate with a seed liquid; wherein the seed liquid is made by inoculating a second growth medium comprising yeast extract, peptone, glucose, maltose and sodium chloride and water with a microbe and culturing said inoculated second growth medium;
culturing said inoculated first growth medium to produce a fermentation broth;
temperature sterilizing the fermentation broth to obtain a sterilized fermentation broth;
filtering the sterilized fermentation broth, to produce a filtrate;
adjusting the pH of the filtrate to an acidic pH to obtain a microbe separation liquid wherein the microbe separation liquid is miscible in an least one organic solvent; and
extracting the microbe separation liquid with the at least one organic solvent, to produce the MEL composition.

16. (canceled)

17. The process of claim 15, wherein the first growth medium comprises:
7% to 20% by weight of the carbon source;
0.5% to 2% by weight of sodium nitrate;
0.1% to 0.3% by weight of yeast extract;
0.3% to 0.7% by weight of potassium dihydrogen phosphate;
0.03% to 0.07% by weight of magnesium sulfate heptahydrate;
0.005% to 0.015% by weight of calcium chloride dihydrate;
0.005% to 0.015% by weight of ferrous sulfate heptahydrate; and
0.0005% to 0.0015% by weight of manganese sulfate;
or
15% by weight of the carbon source;
1% by weight of sodium nitrate;
0.2% by weight of yeast extract;
0.5% by weight of potassium dihydrogen phosphate;
0.05% by weight of magnesium sulfate heptahydrate;
0.01% by weight of calcium chloride dihydrate;
0.01% by weight of ferrous sulfate heptahydrate; and
0.001% by weight of manganese sulfate;
or
10% by weight of the carbon source;
0.5% by weight of sodium nitrate;
0.3% by weight of yeast extract;
0.25% by weight of potassium dihydrogen phosphate;
0.05% by weight of magnesium sulfate heptahydrate;
0.005% by weight of calcium chloride dihydrate;
0.005% by weight of ferrous sulfate heptahydrate; and
0.002% by weight of manganese sulfate;
or
9% by weight of the carbon source;
0.5% by weight of sodium nitrate;
0.3% by weight of yeast extract;
0.25% by weight of potassium dihydrogen phosphate;
0.05% by weight of magnesium sulfate heptahydrate;
0.005% by weight of calcium chloride dihydrate;
0.005% by weight of ferrous sulfate heptahydrate; and
0.002% by weight of manganese sulfate;
or
12% by weight of the carbon source;
0.4% by weight of sodium nitrate;
0.3% by weight of yeast extract;
0.25% by weight of potassium dihydrogen phosphate;
0.05% by weight of magnesium sulfate heptahydrate;
0.005% by weight of calcium chloride dihydrate;
0.005% by weight of ferrous sulfate heptahydrate; and
0.002% by weight of manganese sulfate.

18-21. (canceled)

22. The process of claim 15, wherein the second growth medium comprises:
0.2% to 0.4% by weight of yeast extract;
0.4% to 0.8% by weight of peptone;
0.2% to 0.6% by weight of glucose;
0.05% to 0.15% by weight of maltose; and
0.3% to 0.7% by weight of sodium chloride;
or
0.3% by weight of yeast extract;
0.6% by weight of peptone;
0.4% by weight of glucose;
0.1% by weight of maltose; and
0.5% by weight of sodium chloride.

23-41. (canceled)

42. The process of claim 15, further comprising isolating the organic phase from the extraction of the microbe separation liquid.

43. (canceled)

44. A mannosylerythritol lipid (MEL) composition made by the process of claim 15.

45. A biological pesticide comprising the MEL composition of claim 44.

46. A method of preventing or treating a crop disease caused by a crop pathogen, comprising contacting the crop infected by the crop pathogen with a therapeutically effective amount of the biological pesticide of claim 14.

47. The method of claim 46, wherein the crop disease comprises rice blast, Sorghum hard smut, rice smut, corn leaf blight, and other fungal diseases.

48-49. (canceled)

50. The method of claim 46, wherein the crop is: rice, wheat, corn, or sorghum.
